SEQUENCE LISTING (9 sequences)

SEQ ID NO: 1 - DNA, 4687 bp, Homo sapiens
  CDS (19)..(4179)
cacgagcccaggagagccatgaagcctaggaaagctgagcctcatagcttccgggagaaggttttccggaagaaacctccagtctgtgcagtatgtaaggtgaccatcgatgggacaggcgtttcgtgcagagtctgcaaggtggcgacgcacagaaaatgtgaagcaaaggtgacttcagcctgtcaggccttgcctcccgtggagttgcggcgaaacacggccccagtcaggcgcatagagcacctgggatccaccaaatctctgaaccactcaaagcagcgcagcactctgcccaggagcttcagcctggacccgctcatggagcggcgctgggacttagacctcacctacgtgacggagcgcatcttggccgccgccttccccgcgcggcccgatgaacagcggcaccggggccacctgcgcgagctggcccatgtgctgcaatccaagcaccgggacaagtacctgctcttcaacctttcagagaaaaggcatgacctgacccgcttaaaccccaaggttcaagacttcggctggcctgagctgcatgctccacccctggacaagctgtgctccatctgcaaagccatggagacatggctcagtgctgacccacagcacgtggtcgtactatactgcaagggaaacaagggcaagcttggggtcatcgtttctgcctacatgcactacagcaagatctctgcaggggcggaccaggcactggccactcttaccatgcggaaattctgcgaggacaaggtggccacagaactgcagccctcccagcgtcgatatatcagctacttcagtgggctgctatctggctccatcagaatgaacagcagccctctcttcctgcactatgtgctcatccccatgctgccagcctttgaacctggcacaggcttccagcccttccttaaaatctaccagtccatgcagcttgtctacacatctggagtctatcacattgcaggccctggtccccagcagctttgcatcagcctggagccagccctcctcctcaaaggcgatgtcatggtaacatgttatcacaagggtggccggggcacagaccggaccctcgtgttccgagtccagttccacacctgcaccatccacggaccacagctcactttccccaaggaccagcttgacgaggcctggactgatgagaggttccccttccaagcctccgtggagtttgtcttctcctccagccccgagaagatcaaaggcagcactccacggaacgacccctcggtctctgtcgactacaacaccactgagccagccgtgcgctgggactcctatgagaacttcaaccagcaccacgaggacagtgtggatggctccttgacccacacccggggtcccctggatggcagtccttatgcccaggtgcagcggcctccccggcagacccccccggcaccctctccagagcctccaccaccccccatgctctctgtcagcagcgactcaggccattcctccacgctgaccacagagccggctgctgagtcccctggccggccgccccctacagctgctgaacggcaggagctggatcgcctcctaggaggctgcggagtggccagtgggggccggggagctgggcgcgagacggccatcctagatgacgaagagcagcccactgtgggcggaggcccccacctcggagtgtatccaggccataggcctggcctcagccgccactgctcctgccgccagggctaccgggagccctgcggggttcccaatgggggctactaccggccagagggaaccctggagaggaggcgactggcctacgggggctatgagggatccccccagggctacgccgaggcctcgatggagaagaggcgcctctgccgatcgctgtcagaggggctatacccctacccacctgagatggggaaaccagccactggggactttggctaccgcgccccaggctaccgggaggtggtcatcctggaggaccctgggctgcctgccctatacccatgcccagcctgcgaggagaagctggcgctgcctacagcagccttgtatggactgcggctggagagggaggctggagaagggtgggcaagtgaggctggcaagcctctcctgcacccagtgcggcctgggcacccgctgcctctgctcttgcctgcctgtgggcatcaccatgccccgatgcctgactacagctgcctgaagccacccaaggcaggcgaggaagggcacgagggctgctcctacaccatgtgccccgaaggcaggtatgggcatccagggtaccctgccctggtgacatacagctatggaggagcagttcccagttactgcccagcatatggccgtgtgcctcatagctgtggctctccaggagagggcagagggtatcccagccctggtgcccactccccacgggctggctccatttccccgggcagcccgccctatccacaatctaggaagctgagctacgagatccctacggaggagggaggggacaggtacccattgcctgggcacctggcctcagcaggacctttggcatctgcagagtcgctggagccggtgtcctggagggagggccccagtgggcacagcacactgcctcggtctccccgagatgccccatgcagtgcttcgtcagagttgtctggtccctccacgcccctgcacaccagcagtccagtccagggcaaggaaagcacccggcgacaggacaccaggtcccccacctcagcgcccactcagagactgagtcctggcgaggccttgccccctgtttcccaggcaggcaccggaaaggcccctgagctgccgtcgggaagtgggcctgagcctctggcccctagcccagtctctccgaccttccctcccagctcgcccagtgactggcctcaggaaaggagtccagggggccactcagatggcgccagtcctcggagccctgtgcccaccacacttcctggcctccgccacgccccctggcaaggccctcgaggcccccccgacagcccagatgggtctcccctcactcctgtgccttcccagatgccctggcttgtggccagcccagagccgcctcagagctcacctacacctgctttccccctggctgcctcctatgacaccaatggccttagccagcccccacttcctgagaaacgccacctgcccgggccggggcaacagccaggaccctggggcccagagcaggcatcatcgccagccagaggcatcagtcaccatgtcaccttcgcacctctgctctcagataatgtcccccaaaccccagagcctcctacacaagagagccaaagcaatgtcaagtttgtccaggatacatccaagttctggtacaagccacacctgtcccgtgaccaagccattgccctgctgaaggacaaggaccctggggccttcctgatcagggacagtcattcattccaaggagcttatgggctggccctcaaggtggccacaccgccacccagtgcccagccctggaaaggggaccccgtggaacagctggtccgccatttcctcatcgagactgggcccaaaggggtgaagatcaagggctgccccagtgagccctactttggcagcctgtccgccttggtctcccagcactccatctcccccatctccctgccctgctgcctgcgcattctcagcaaagatcctctggaagagaccccagaggctccagtgcccaccaacatgagcacagcggcagacctcctgcgtcagggtgctgcctgcagcgtgctctacttgacctcagtggagacagagtcactgacgggcccccaagctgtggcccgggccagctctgcagctctgagctgtagcccccgcccgacaccagctgttgtccacttcaaggtgtcagcccagggcattacactgacggacaaccaaaggaagctcttctttcgccgccattatccagtgaacagcatcaccttctccagcactgaccctcaagaccggagatggaccaacccagacgggaccacctccaagatctttggtttcgtggccaagaagccgggaagcccctgggagaatgtgtgtcacctctttgcagagcttgacccagatcagcctgctggcgccattgtcaccttcatcaccaaagttctactgggccagagaaaatgaaggaaggccacaagctcagagcccacatcaacactgcccccctcccagcaccccacagccctcacatcccctggcctggacccaggagacccaggagaaagcaccctcccttaggaatgaggagtgggcatcaggcctgggacactgctctccttccccgcccccagcctgctaagttaagtggacaggcccacaagatgaccttgcatgtgagcagatggcagagatgggtgtgtgaggggtgaggaggcatcagcagttgagccccgaaggagatcaggcagccccacctgcaggagaacgtcagccctccaggggatcagcccctgccagttccacccagctgcaggtgccagcacggcagggatgggagaggggtggggagcgagtcactgcctcctctgagcagagattcagagtaggatcacatgaataggggaaaaaagagagtctatttttgtctaataataaagaatttctataaactttaaaaaaaaaaaaaaaaaaa

SEQ ID NO: 2 - protein, 1386 aa, Homo sapiens
MKPRKAEPHSFREKVFRKKPPVCAVCKVTIDGTGVSCRVCKVATHRKCEAKVTSACQALPPVELRRNTAPVRRIEHLGSTKSLNHSKQRSTLPRSFSLDPLMERRWDLDLTYVTERILAAAFPARPDEQRHRGHLRELAHVLQSKHRDKYLLFNLSEKRHDLTRLNPKVQDFGWPELHAPPLDKLCSICKAMETWLSADPQHVVVLYCKGNKGKLGVIVSAYMHYSKISAGADQALATLTMRKFCEDKVATELQPSQRRYISYFSGLLSGSIRMNSSPLFLHYVLIPMLPAFEPGTGFQPFLKIYQSMQLVYTSGVYHIAGPGPQQLCISLEPALLLKGDVMVTCYHKGGRGTDRTLVFRVQFHTCTIHGPQLTFPKDQLDEAWTDERFPFQASVEFVFSSSPEKIKGSTPRNDPSVSVDYNTTEPAVRWDSYENFNQHHEDSVDGSLTHTRGPLDGSPYAQVQRPPRQTPPAPSPEPPPPPMLSVSSDSGHSSTLTTEPAAESPGRPPPTAAERQELDRLLGGCGVASGGRGAGRETAILDDEEQPTVGGGPHLGVYPGHRPGLSRHCSCRQGYREPCGVPNGGYYRPEGTLERRRLAYGGYEGSPQGYAEASMEKRRLCRSLSEGLYPYPPEMGKPATGDFGYRAPGYREVVILEDPGLPALYPCPACEEKLALPTAALYGLRLEREAGEGWASEAGKPLLHPVRPGHPLPLLLPACGHHHAPMPDYSCLKPPKAGEEGHEGCSYTMCPEGRYGHPGYPALVTYSYGGAVPSYCPAYGRVPHSCGSPGEGRGYPSPGAHSPRAGSISPGSPPYPQSRKLSYEIPTEEGGDRYPLPGHLASAGPLASAESLEPVSWREGPSGHSTLPRSPRDAPCSASSELSGPSTPLHTSSPVQGKESTRRQDTRSPTSAPTQRLSPGEALPPVSQAGTGKAPELPSGSGPEPLAPSPVSPTFPPSSPSDWPQERSPGGHSDGASPRSPVPTTLPGLRHAPWQGPRGPPDSPDGSPLTPVPSQMPWLVASPEPPQSSPTPAFPLAASYDTNGLSQPPLPEKRHLPGPGQQPGPWGPEQASSPARGISHHVTFAPLLSDNVPQTPEPPTQESQSNVKFVQDTSKFWYKPHLSRDQAIALLKDKDPGAFLIRDSHSFQGAYGLALKVATPPPSAQPWKGDPVEQLVRHFLIETGPKGVKIKGCPSEPYFGSLSALVSQHSISPISLPCCLRILSKDPLEETPEAPVPTNMSTAADLLRQGAACSVLYLTSVETESLTGPQAVARASSAALSCSPRPTPAVVHFKVSAQGITLTDNQRKLFFRRHYPVNSITFSSTDPQDRRWTNPDGTTSKIFGFVAKKPGSPWENVCHLFAELDPDQPAGAIVTFITKVLLGQRK

SEQ ID NO: 3 - DNA, 1146 bp, Homo sapiens
  CDS (1)..(1146)
ctgaaccactcaaagcagcgcagcactctgcccaggagcttcagcctggacccgctcatggagcggcgctgggacttagacctcacctacgtgacggagcgcatcttggccgccgccttccccgcgcggcccgatgaacagcggcaccggggccacctgcgcgagctggcccatgtgctgcaatccaagcaccgggacaagtacctgctcttcaacctttcagagaaaaggcatgacctgacccgcttaaaccccaaggttcaagacttcggctggcctgagctgcatgctccacccctggacaagctgtgctccatctgcaaagccatggagacatggctcagtgctgacccacagcacgtggtcgtactatactgcaagggaaacaagggcaagcttggggtcatcgtttctgcctacatgcactacagcaagatctctgcaggggcggaccaggcactggccactcttaccatgcggaaattctgcgaggacaaggtggccacagaactgcagccctcccagcgtcgatatatcagctacttcagtgggctgctatctggctccatcagaatgaacagcagccctctcttcctgcactatgtgctcatccccatgctgccagcctttgaacctggcacaggcttccagcccttccttaaaatctaccagtccatgcagcttgtctacacatctggagtctatcacattgcaggccctggtccccagcagctttgcatcagcctggagccagccctcctcctcaaaggcgatgtcatggtaacatgttatcacaagggtggccggggcacagaccggaccctcgtgttccgagtccagttccacacctgcaccatccacggaccacagctcactttccccaaggaccagcttgacgaggcctggactgatgagaggttccccttccaagcctccgtggagtttgtcttctcctccagccccgagaagatcaaaggcagcactccacggaacgacccctcggtctctgtcgactacaacaccactgagccagccgtgcgctgggactcctatgagaacttcaaccagcaccacgaggacagtgtggatggctccttgacccacacccggggtcccctggatggcagtccttatgcccaggtgcag

SEQ ID NO: 4 - protein, 382 aa, Homo sapiens
LNHSKQRSTLPRSFSLDPLMERRWDLDLTYVTERILAAAFPARPDEQRHRGHLRELAHVLQSKHRDKYLLFNLSEKRHDLTRLNPKVQDFGWPELHAPPLDKLCSICKAMETWLSADPQHVVVLYCKGNKGKLGVIVSAYMHYSKISAGADQALATLTMRKFCEDKVATELQPSQRRYISYFSGLLSGSIRMNSSPLFLHYVLIPMLPAFEPGTGFQPFLKIYQSMQLVYTSGVYHIAGPGPQQLCISLEPALLLKGDVMVTCYHKGGRGTDRTLVFRVQFHTCTIHGPQLTFPKDQLDEAWTDERFPFQASVEFVFSSSPEKIKGSTPRNDPSVSVDYNTTEPAVRWDSYENFNQHHEDSVDGSLTHTRGPLDGSPYAQVQ

SEQ ID NO: 5 - DNA, 4071 bp, Homo sapiens
  CDS (1)..(4071)
atggagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaaatcattattctagtaggcacggcggtgattgccatgttcttctggctacttcttgtcatcatcctacggaccgttaagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaa

SEQ ID NO: 6 - protein, 1356 aa, Homo sapiens [sequence omitted]

SEQ ID NO: 7 - DNA, 1704 bp, Homo sapiens
  CDS (1)..(1704)
aagcgggccaatggaggggaactgaagacaggctacttgtccatcgtcatggatccagatgaactcccattggatgaacattgtgaacgactgccttatgatgccagcaaatgggaattccccagagaccggctgaagctaggtaagcctcttggccgtggtgcctttggccaagtgattgaagcagatgcctttggaattgacaagacagcaacttgcaggacagtagcagtcaaaatgttgaaagaaggagcaacacacagtgagcatcgagctctcatgtctgaactcaagatcctcattcatattggtcaccatctcaatgtggtcaaccttctaggtgcctgtaccaagccaggagggccactcatggtgattgtggaattctgcaaatttggaaacctgtccacttacctgaggagcaagagaaatgaatttgtcccctacaagaccaaaggggcacgattccgtcaagggaaagactacgttggagcaatccctgtggatctgaaacggcgcttggacagcatcaccagtagccagagctcagccagctctggatttgtggaggagaagtccctcagtgatgtagaagaagaggaagctcctgaagatctgtataaggacttcctgaccttggagcatctcatctgttacagcttccaagtggctaagggcatggagttcttggcatcgcgaaagtgtatccacagggacctggcggcacgaaatatcctcttatcggagaagaacgtggttaaaatctgtgactttggcttggcccgggatatttataaagatccagattatgtcagaaaaggagatgctcgcctccctttgaaatggatggccccagaaacaatttttgacagagtgtacacaatccagagtgacgtctggtcttttggtgttttgctgtgggaaatattttccttaggtgcttctccatatcctggggtaaagattgatgaagaattttgtaggcgattgaaagaaggaactagaatgagggcccctgattatactacaccagaaatgtaccagaccatgctggactgctggcacggggagcccagtcagagacccacgttttcagagttggtggaacatttgggaaatctcttgcaagctaatgctcagcaggatggcaaagactacattgttcttccgatatcagagactttgagcatggaagaggattctggactctctctgcctacctcacctgtttcctgtatggaggaggaggaagtatgtgaccccaaattccattatgacaacacagcaggaatcagtcagtatctgcagaacagtaagcgaaagagccggcctgtgagtgtaaaaacatttgaagatatcccgttagaagaaccagaagtaaaagtaatcccagatgacaaccagacggacagtggtatggttcttgcctcagaagagctgaaaactttggaagacagaaccaaattatctccatcttttggtggaatggtgcccagcaaaagcagggagtctgtggcatctgaaggctcaaaccagacaagcggctaccagtccggatatcactccgatgacacagacaccaccgtgtactccagtgaggaagcagaacttttaaagctgatagagattggagtgcaaaccggtagcacagcccagattctccagcctgactcggggaccacactgagctctcctcctgtttaa

SEQ ID NO: 8 - protein, 567 aa, Homo sapiens
KRANGGELKTGYLSIVMDPDELPLDEHCERLPYDASKWEFPRDRLKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGNLLQANAQQDGKDYIVLPISETLSMEEDSGLSLPTSPVSCMEEEEVCDPKFHYDNTAGISQYLQNSKRKSRPVSVKTFEDIPLEEPEVKVIPDDNQTDSGMVLASEELKTLEDRTKLSPSFGGMVPSKSRESVASEGSNQTSGYQSGYHSDDTDTTVYSSEEAELLKLIEIGVQTGSTAQILQPDSGTTLSSPPV

SEQ ID NO: 9 - DNA, 606 bp, Homo sapiens
gaaacgccacctgcccgggccggggcaacagccaggaccctggggcccagagcaggcatcatcgccagccagaggcatcagtcaccatgtcaccttcgcacctctgctctcagataatgtcccccaaaccccagagcctcctacacaagagagccaaagcaatgtcaagtttgtccaggatacatccaagttctggtacaagccacacctgtcccgtgaccaagccattgccctgctgaaggacaaggaccctggggccttcctgatcagggacagtcattcattccaaggagcttatgggctggccctcaaggtggccacaccgccacccagtgcccagccctggaaaggggaccccgtggaacagctggtccgccatttcctcatcgagactgggcccaaaggggtgaagatcaagggctgccccagtgagccctactttggcagcctgtccgccttggtctcccagcactccatctcccccatctccctgccctgctgcctgcgcattctcagcaaagatcctctggaagagaccccagaggctccagtgcccaccaacatgagcacagcggcagacctcctgcgtcagggtgctgcctgcag

(19) United States
(12) Patent Application Publication
(10) Pub. No.: US 2003/0158083 A1
(43) Pub. Date: Aug. 21, 2003

(54) Method of effecting angiogenesis by modulating the function of a novel endothelia phosphatase

(75) Inventor: Kevin Gene Peters, Loveland, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/327,414

(22) Filed: Dec. 20, 2002

Related U.S. Application Data
(60) Provisional application No. 60/355,125, filed on Feb. 8, 2002.

(51) Int. Cl. A61K 31/00; G01N 33/574; C07H 21/04; A61K 38/46; C12N 9/16

(52) U.S. Cl. 514/1; 424/94.6; 435/196; 435/320.1; 435/325; 435/7.23; 536/23.2

(57) ABSTRACT

KDR associated phosphatase (KAPh) is useful as a target to screen for agents useful for the treatment of angiogenesis mediated disorders.

### CROSS REFERENCE TO RELATED APPLICATIONS

This application claims priority under Title 35, United States Code 119(e) from Provisional Application Serial No. 60/355,125 filed Feb. 8, 2002.

### FIELD OF INVENTION

This invention is directed to KAPh and its use to screen agents useful in the treatment angiogenesis mediated disorders.

### BACKGROUND OF THE INVENTION

Angiogenesis, the sprouting of new blood vessels from the pre-existing vasculature, plays a crucial role in a wide range of physiological and pathological processes in Nguyen, L. L. et al, Int. Rev. Cytol., 204, 1-48, (2001). It is a complex process that is mediated by communication between the endothelial cells that line blood vessels and their surrounding environment, Glienke, J. et al, Eur. J. Biochem., 267, 2820-2830, (2000). In the early stages of angiogenesis, tissue or tumor cells produce and secrete pro-angiogenic growth factors in response to environmental stimuli such as hypoxia, Bussolino, F., Trends Biochem. Sci., 22, 251-256, (1997). These factors diffuse to nearby endothelial cells and stimulate receptors that lead to the production and secretion of proteases that degrade the surrounding extracellular matrix, Raza, S. L. et al, J. Investig. Dermatol. Symp. Proc., 5, 47-54, (2000); Stetler-Stevenson, W. G., Surg. Oncol. Clin. N. Am., 10, 383-392, (2001). These activated endothelial cells begin to migrate and proliferate into the surrounding tissue toward the source of these growth factors, Bussolino, F., Trends Biochem. Sci., 22, 251-256, (1997). Endothelial cells then stop proliferating and differentiate into tubular structures, which is the first step in the formation of stable, mature blood vessels, Glienke, J. et al, Eur. J. Biochem., 267, 2820-2830, (2000). Subsequently, periendothelial cells, such as pericytes and smooth muscle cells, are recruited to the newly formed vessel in a further step toward vessel maturation. or exacerbate an existing pathological condition. For example, ocular neovascularization has been implicated as the most common cause of blindness and underlies the pathology of approximately 20 eye diseases. In certain previously existing conditions such as arthritis, newly formed capillary blood vessels invade the joints and destroy cartilage. In diabetes, new capillaries formed in the retina invade the vitreous humor, causing bleeding and blindness.

Although many disease states are driven by persistent unregulated angiogenesis, many disease states could be treated by increased angiogenesis. Tissue growth and repair are biologic systems wherein cellular proliferation and angiogenesis occur. Thus an important aspect of wound repair is the revascularization of damaged tissue by angiogenesis.

Impaired tissue healing is a significant problem in health care. Chronic, non-healing wounds are a major cause of prolonged morbidity in the aged human population. This is especially the case in bedridden or diabetic patients who develop severe, non-healing skin ulcers. In many of these cases, the delay in healing is a result of inadequate blood supply either as a result of continuous pressure or of vascular blockage. Poor capillary circulation due to small artery atherosclerosis or venous stasis contribute to the failure to repair damaged tissue. Such tissues are often infected with microorganisms that proliferate unchallenged by the innate defense systems of the body which require well vascularized tissue to effectively eliminate pathogenic organisms. As a result, most therapeutic intervention centers on restoring blood flow to ischemic tissues thereby allowing nutrients and immunological factors access to the site of the wound.

Atherosclerotic lesions in large vessels can cause tissue ischemia that could be ameliorated by modulating blood vessel growth to supply the affected tissue. For example, atherosclerotic lesions in the coronary arteries cause angina and myocardial infarction that could be prevented if one could restore blood flow by stimulating the growth of collateral arteries. Similarly, atherosclerotic lesions in the large arteries that supply the legs cause ischemia in the skeletal muscle that limits mobility and in some cases necessitates amputation which could also be prevented by improving blood flow with angiogenic therapy.

Other diseases such as diabetes and hypertension are characterized by a decrease in the number and density of small blood vessels such as arterioles and capillaries. These small blood vessels are critical for the delivery of oxygen and nutrients and any decrease in the number and density of these vessels contributes to the adverse consequences of hypertension and diabetes including claudication, ischemic ulcers, accelerated hypertension, and renal failure. These common disorders and many other less common ailments such as Burgers disease would be ameliorated by increasing the number and density of small blood vessels using angiogenic therapy.

In view of the foregoing, there is a need to identify biochemical targets in the treatment of angiogenesis modulated disorders. One important mediator of angiogenesis is the endothelial cell-specific mitogen, vascular endothelial growth factor (VEGF) (Keck, P J et al., Science 1989; 246(4935):1309-1312; Leung D W, et al., Science 1989; 246(4935): 1306-1309).

The biological activity of VEGF is mediated through its interaction with two high affinity receptor tyrosine kinases, vascular endothelial growth factor receptor-1 (VEGFR-1) (Flt-1, fms-like tyrosine kinase) and vascular endothelial growth factor receptor-2 (VEGFR2) (human KDR, kinase-insert domain-containing receptor/murine Flk-1, fetal liver kinase-1) (Shibuya M., et al., Oncogene 1990; 5(4):519-524; de Vries C, et al., Science 1992; 255(5047):989-991; Peters K G, et al., Proc Natl Acad Sci U S A 1993; 90(19):8915-8919; Matthews W, et al., Proc Natl Acad Sci U S A 1991; 88(20):9026-9030; Terman B I, et al., Biochem Biophys Res Commun 1992; 187(3):1579-1586). VEGF also binds neuropilin (Npn-1) forming a co-receptor complex with VEGFR2 which may account for differences in signaling by various VEGF isoforms (Gagnon M L, et al., Proc Natl Acad Sci U S A 2000; 97(6):2573-2578; Whitaker G B, et al., J Biol Chem 2001; 276(27):25520-25531). The importance of the VEGF receptors, Flt-1 and KDR/Flk-1, in vascular development was confirmed by the generation of null mice (Fong G H, et al., Nature 1995; 376(6535):66-70; Shalaby F, et al., Cell 1997; 89(6):981-990). Although, these knock-out studies yielded somewhat different vascular phenotypes for each receptor, mouse embryos null for either receptor died in utero between days 8.5 and 9.5.

The binding of VEGF to its cognate receptors triggers the activation of intrinsic receptor tyrosine kinase activity that results in their autophosphorylation (Ullrich A, et al., Cell 1990; 61(2):203-212). Of the two VEGF receptors, there is growing evidence that VEGFR2 (KDR/Flk-1) is the principal receptor responsible for mediating the mitogenic activity of VEGF (Park J E, et al., J Biol Chem 1994; 269(41):25646-25654; Keyt B A, et al., J Biol Chem 1996; 271(10):5638-5646; Carmeliet P, et al., Nat Med 2001; 7(5):575-583). Moreover, it has been shown that following VEGF stimulation, KDR undergoes strong ligand-dependent tyrosine autophosphorylation, whereas Fit-i has a much weaker response (Waltenberger J, et al., J Biol Chem 1994; 269(43):26988-26995; Seetharam L, et al., 1995; 10(1):135-147). This VEGF-induced autophosphorylation of specific tyrosine residues within the intracellular kinase domain of KDR provides functional docking sites for the receptor to form protein-protein interactions with Src homology 2 (SH2) domain-containing proteins. These cytoplasmic signaling molecules directly link the activated receptor to signal transduction cascades and ultimately lead to cellular responses (Moran M F, et al., Proc Natl Acad Sci U S A 1990; 87(21):8622-8626; Cantley L C, et al., Cell 1991; 64(2):281-302). To date several KDR-binding proteins have been identified, including phospholipase Cγ (PLCγ), a Shc-related adaptor protein (Sck), a low molecular weight protein tyrosine phosphatase (HCPTPA), VEGF receptor-associated protein (VRAP), and a cytoplasmic protein tyrosine phosphatase (SHP-1) (Cunningham S A, et al., Biochem Biophys Res Commun 1997; 240(3):635-639; Igarashi K, et al., Biochem Biophys Res Commun 1998; 251(1):77-82; Warner A J, et al., Biochem J 2000; 347(Pt 2):501-509; Huang L, et al., J Biol Chem 1999; 274(53):38183-38188; Wu L W, et al., J Biol Chem 2000; 275(9):6059-6062; Guo D Q, et al., J Biol Chem 2000; 275(15):11216-11221).

The cytoplasmic domain of KDR contains nineteen tyrosine residues which, if phosphorylated, could serve as potential docking sites for signaling molecules. Although, tyrosine residues 951, 996, 1054, and 1059 have been identified as autophosphorylation sites for KDR, those residues critical for transducing the biological activity of VEGF have yet to be precisely determined (Dougher-Vermazen M, et al., Biochem Biophys Res Commun 1994; 205(1):728-738; Dougher M, et al., Oncogene 1999; 18(8):1619-1627; Takahashi T, et al., EMBO J 2001; 20(11):2768-2778). Regardless, it is clear that tyrosine phosphorylation of the cytoplasmic domain of KDR plays an important role in recruiting signaling molecules to the receptor following stimulation with VEGF. Thus, there is a continuing need to identify modulators of angiogenesis, specifically modulators of VEGFR2.

### SUMMARY OF THE INVENTION

The present invention is based on the surprising discovery of the novel protein KDR Associated Phosphatase (KAPh) as a modulator of angiogenesis mediated disorders.

One aspect of the invention provides for an isolated KAPh or variant thereof. In one embodiment, the isolated KAPh is SEQ ID NO 1.

Another aspect of the invention provides for the use of KAPh in the treatment of an angiogenesis mediated disorder. In one embodiment, the method is directed to the use of KAPh in the treatment of a VEGFR2 mediated disorder.

Another aspect provides for an isolated nucleotide sequence encoding a KAPh or variant thereof. One embodiment provides a polynucleotide that hybridizes to and which is at least 80% complimentary to a polynucleotide encoding a polypeptide of SEQ ID NO 2. One embodiment provides an expression vector comprising a promoter sequence operably linked to a nucleotide encoding KAPh. Another embodiment provides a host cell comprising a promoter sequence operably linked to a nucleotide encoding KAPh. Still another embodiment provides a method for producing a KAPh protein, said method comprising: (a) culturing a host cell comprising a promoter sequence operably linked to a nucleotide encoding a KAPh protein; and (b) recovering the KAPh protein.

One aspect of the invention provides for a method of screening an agent useful for treating an angiogenesis mediated disorder, suitable for high throughput screening, comprising the steps of: (a) exposing KAPh to the agent; and (b) measuring activity of KAPh; wherein a modulation in KAPh activity indicates the agent is useful for treating the angiogenesis mediated disorder.

Another aspect of the invention provides for a method of screening an agent useful for modulating angiogenesis comprising the steps of: (a) exposing KAPh to the agent; (b) exposing KAPh to VEGFR2; and (c) measuring association of KAPh and VEGFR2; wherein a modulation in the association of KAPh and VEGFR2 indicates the agent is useful for treating the angiogenesis mediated disorder.

One aspect of the invention provides for a method of screening an agent useful for treating an angiogenesis mediated disorder comprising the steps of: (a) exposing a KAPh encoding nucleotide sequence to the agent; (b) measuring association of the agent to the KAPh encoding nucleotide; wherein the association of the agent to the KAPh encoding nucleotide indicates the agent is useful for treating the angiogenesis mediated disorder.

One aspect of the invention provides for a method of screening an agent useful for treating an angiogenesis mediated disorder, suitable for a cell-based assay, comprising the steps of: (a) exposing a cell to the agent; and (b) measuring activity of KAPh in the cell; wherein a modulation in the activity of KAPh indicates the agent is useful for treating the angiogenesis mediated disorder.

Another aspect of the invention provides for a method of screening an agent useful for treating an angiogenesis mediated disorder, suitable for a cell-based assay, comprising the steps of: (a) exposing a cell to the agent; and (b) measuring expression of KAPh in the cell; wherein a modulation in the expression of KAPh indicates the agent is useful for treating the angiogenesis mediated disorder.

Another aspect of the invention provides for a method of screening an agent useful for treating an angiogenesis mediated disorder comprising the steps of: (a) exposing a cell to the agent; and (b) measuring the association of KAPh and VEGFR2; wherein a modulation in the association of KAPh and VEGFR2 indicates the agent is useful for treating the angiogenesis mediated disorder.

One aspect of the invention provides for an isolated antibody that binds to KAPh. Another aspect of the invention provides for an isolated antibody that binds to KAPh, wherein the binding modulates KAPh activity, or KAPh expression, or KAPh and VEGFR2 association. In one embodiment, the antibody binds to an epitope selected from amino acids 1054-1386; 431-446; and 1123-1386 of SEQ ID NO 2.

One aspect of the invention provides for a pharmaceutical composition comprising: (a) a safe and effective amount of KAPh or a nucleotide encoding the same; and (b) a pharmaceutically-acceptable carrier.

Another aspect of the invention provides for a pharmaceutical composition comprising: (a) a safe and effective amount of an agent that modulates KAPh activity, or KAPh expression, or KAPh and VEGFR2 association; and (b) a pharmaceutically-acceptable carrier.

Another aspect of the invention provides for a method of treating an angiogenesis mediated disorder in a subject in need thereof by administering a safe and effective amount of an agent that modulates KAPh activity, or KAPh expression, or KAPh and VEGFR2 association.

### BRIEF DESCRIPTION OF THE FIGURES

FIG. 1. Identification of a novel protein KAPh as a VEGF receptor-2 interactor by the yeast two-hybrid system. Specific, autophosphorylation-dependent interaction of a human endothelial cell library-encoded protein with VEGF R-2. Six colonies from each transformation (horizontal rows of colonies) were picked and patched onto (A) histidine-containing media (lacking leucine and tryptophan) to confirm the successful transformation of each combination of yeast expression vectors. Yeast colonies were then replica plated onto (B) histidine-deficient media to test for activation of the HIS3 reporter gene and onto (C) uracil-deficient media to test for activation of the URA3 reporter gene. (row 1) pDB Leu ‘empty bait’+pPC 86 ‘empty prey’, (row 2) pDB Leu ‘empty bait’+KAPh SH2 domain fragment, (row 3) VEGF R-2 wild-type +KAPh SH2 domain fragment, (row 4) VEGF R-2 (K870R)+KAPh SH2 domain fragment, (row 5) VEGF R-2 wild-type+pPC 86 ‘empty prey’, (row 6) VEGF R-2 (K870R)+pPC 86 ‘empty prey’. (D) Yeast expressing the pDB Leu VEGF R-2 wild-type or mutant K870R and pPC 86 KAPh SH2 domain fragment were grown in liquid cultures for 16 h at 30° C. Activation of the LacZ reporter was monitored using a β-galactosidase assay. Results are presented as the mean±S.D. of triplicate independent cultures.

FIG. 2. Autophosphorylation-dependent association of KAPh with the human VEGF receptor-2. In vitro GST pull-down analyses, (A) baculovirus-expressed recombinant human wild-type (lane 5) and catalytically inactive (K870R, lane 7) GST VEGF R-2 fusion proteins and GST (lane 3) were incubated with HEK 293 cell lysates expressing 6×His-tagged KAPh SH2 domain. Complexes immobilized on glutathione sepharose were washed, separated by SDS-PAGE and visualized by autoradiography with an anti-HisG antibody. The input (lane 1) represents 10% of the protein in the binding assay. The immunoblot was sequentially stripped of antibody and re-probed with anti-GST and anti-phosphotyrosine antibodies. (B) Similarly, baculovirus-expressed recombinant wild-type and catalytically inactive GST fusion proteins of the human VEGF receptor-1 (WT, lane 3; K862R, lane 4), VEGF receptor-2 (WT, lane 5; K870R, lane 6), Tie1 (WT, lane 10; K870R, lane 11), Tie2/TEK (WT, lane 12; K870R, lane 13), and GST (lanes 2 and 9) were incubated with HEK 293 cell lysates expressing 6×His-tagged KAPh full-length protein. Complexes immobilized on glutathione sepharose were washed, separated by SDS-PAGE and visualized by autoradiography with an anti-HisG antibody. The input (lanes 1 and 8) represents 10% of the protein in the binding assay. The immunoblot was sequentially stripped of antibody and re-probed with anti-GST and anti-phosphotyrosine antibodies. (C) Similarly, baculovirus-expressed recombinant wild-type (WT, lane 2) and mutant (K870R, lane 3; Y951F, lane 4; Y1175F, lane 5) GST fusion proteins of the human VEGF receptor-2 were incubated with HEK 293 cell lysates expressing 6×His-tagged KAPh full-length protein. Complexes immobilized on glutathione sepharose were washed, separated by SDS-PAGE and visualized by autoradiography with an anti-HisG antibody. The input (lane 1) represents 10% of the protein in the binding assay. The immunoblot was sequentially stripped of antibody and re-probed with anti-GST and anti-phosphotyrosine anti bodies. Co-immunoprecipitation analyses, (D) VEGF receptor-2 immunoprecipitates (IP) from unstimulated (lanes 2 and 5) and VEGF-stimulated (lanes 3, 4, and 6) cells which transiently expressed 6×His-tagged full-length KAPh (lanes 4-6) domain were probed by immunoblot with an anti-HisG antibody. A mock IP (no 1° VEGF R-2 antibody) is shown in lane 4. The input (lane 1) represents roughly 10% of the protein in the binding assay.

FIG. 3. Schematic Illustration of KAPh functional domains. The translated KAPh cDNA was analyzed by SMART and Pfam databases to reveal functional domains. The C1/DAG domain (amino acids 9-56 of SEQ ID NO: 2) has been shown in other proteins to be important for binding phorbolesters and diacylglycerol. The PTP/DSP domain (amino acids 166-287 of SEQ ID NO: 2) is found in proteins containing protein tyrosine phosphatase or dual specificity phosphatase catalytic domains. The SH2 domain (amino acids 1117-1215 of SEQ ID NO: 2) is a Src homology 2 domain that has been shown to bind phosphotyrosine-containing polypeptides in other proteins. PTB domain (amino acids 1249-1386 of SEQ ID NO: 2) is a phosphotyrosine-binding domain that has been shown to facilitate interaction with various activated tyrosine-phosphorylated receptors. Those skilled in the art are aware that the amino acid boundaries for protein functional domains are based on homology and thus vary in size from protein to protein.

FIG. 4. Expression and tissue distribution of KAPh mRNA and protein. (A) top, KAPh mRNA transcript in various human tissues. A preblotted membrane containing approximately 2 μg of poly(A)⁺ RNA per lane from twelve different human tissues was obtained from Clontech and was hybridized with a random primed ³²P-labeled human KAPh cDNA probe. Bottom, the blot was stripped and rehybridized with a β-actin probe to evaluate the relative amounts of RNA on the blot. The 4.7 kb transcript is indicated by an arrow. (B) top, KAPh mRNA transcript in various mouse tissues. A preblotted membrane containing approximately 2 μg of poly(A)⁺ RNA per lane from twelve different mouse tissues was obtained from OriGene Technologies, Inc. and was hybridized with a random primed ³²P-labeled mouse KAPh cDNA probe. Bottom, the blot was stripped and rehybridized with a β-actin probe to evaluate the relative amounts of RNA on the blot. The 4.7 kb transcript is indicated by an arrow. (C) top, KAPh mRNA transcript in various stages of mouse embryo development. A preblotted membrane containing approximately 2 μg of poly(A)⁺ RNA per lane from four different stages of mouse embryo development was obtained from Clontech and was hybridized with a random primed ³²P-labeled mouse KAPh cDNA probe. Bottom, the blot was stripped and rehybridized with a β-actin probe to evaluate the relative amounts of RNA on the blot. The 4.7 kb transcript is indicated by an arrow. (D) Expression of KAPh protein in lysates of human umbilical vein endothelial cells (HUVECs). A polyclonal KAPh antibody was incubated with HEK 293 cell lysates expressing 6×His-tagged KAPh full-length protein (lane 1) and HUVEC whole cell lysate (lane 3), no protein was loaded in lane 2. A specific band with an apparent molecular weight of ˜160 kDa (lane 3) corresponding to the endogenous KAPh present in endothelial cells was recognized by the antibody. The KAPh antibody also recognized the overexpressed, epitope-tagged full-length KAPh (lane 1). As expected, due to the presence of the epitope tag, recombinant 6 x His-tagged KAPh appeared to migrate as a slightly larger protein.

FIG. 5. Localization of KAPh expression in the vasculature. Normal adult rat tissues were probed by immunohistochemistry (IHC) with anti-Tie2 and anti-KAPh antibodies to determine the expression pattern of KAPh. Snap-frozen, post-fixed rat heart sections probed with anti-Tie2, used as endothelium-selective marker, showed strong expression of Tie2 in the vascular endothelium (indicated with arrows) [panels A (X20) and C (X100)]. KAPh showed distinct expression in the lumen of vessels indicative of vascular endothelial staining [panels B (X20) and D (X100)]. Furthermore, additional studies were performed using snap-frozen, post-fixed rat kidney sections. Tie2 showed strong expression in fenestrated glomerular capillaries (GL) and showed expression restricted to the vascular endothelium [panels E (X20) and G (X100)]. KAPh was also expressed in glomerular capillaries (GL) and the vascular endothelium, however, KAPh was also expressed in the layers of smooth muscle which surround larger blood vessels [panels F (X20) and H (X100)].

FIG. 6 Comparison of phosphatase activity of KAPh and PTEN as determined using a fluorogenic substrate (DiFMUP). (A) Purified, recombinant KAPh phosphatase domain (amino acids 83-464) and full-length PTEN (amino acids 1-403) were each mixed with a fluorogenic phosphatase substrate (DiFMUP) in assay buffer in a 384-well microplate. Following incubation at ambient temperature for 15 or 60 min., the fluorescence intensity of the liberated product (DiFMU) was measured using a Victor 5 microplate reader. The results are plotted as mean±S.D. of triplicate independent wells and used as an assessment of phosphatase activity. (B) Alignment of amino acid residues which comprise the phosphatase active site of KAPh and PTEN. Several important residues, including an invariant cysteine (boxed in gray) that is absolutely required for catalysis, are present in both KAPh (Cys-208) and PTEN (Cys-124). Interestingly, both KAPh and PTEN contain basic residues (boxed) at the critical +1, +4, and +6 positions suggesting that they may dephosphorylate a common pool of substrates.

### SEQUENCE LISTING DESCRIPTION

Each of the nucleotide and protein sequences in the sequence listing, along with the corresponding Genbank or Derwent accession number(s) and animal species from which it is cloned, is shown in Table I.

TABLE I

| | | | | Related |
|---|---|---|---|---|
| | | | Genbank (GB) | Genbank |
| | | | or Derwent (D) | (GB) or |
| Sequence | SEQ ID NO: | | Accession No. | Derwent (D) |
| Descrip- | Nucleotide, | | for Nucleotide | Accession |
| tion | Amino Acid | Species | Sequence | Nos. |
| KAPh | 1, 2 | Homo Sapiens | | |
| KAPh | 3, 4 | Homo Sapiens | | |
| catalytic | | | | |
| domain | | | | |
| KDR | 5, 6 | Homo Sapiens | AF063658 | NM_002019 |
| (VEGFR2, | | | | |
| Flk1) | | | | |
| VEGFR2 | 7, 8 | Homo sapiens | AF063658 | |
| Intra- | | | | |
| cellular | | | | |
| region | | | | |
| containing | | | | |
| kinase | | | | |
| domain | | | | |

### DETAILED DESCRIPTION OF THE INVENTION

I. Definitions.

“Angiogenesis,” as used herein, means the formation of new blood vessels from pre-existing vasculature.

“Modulate angiogenesis,” as used herein, means to modify angiogenesis. The modulation of angiogenesis encompasses both the stimulation and inhibition of angiogenesis. As used herein, “stimulation of angiogenesis,” means to beneficially enhance or augment a naturally occurring angiogenic process or, alternatively, induce or initiate an angiogenic process. As used herein, “inhibition of angiogenesis,” means to beneficially reduce or diminish either a naturally occurring angiogenic process or disease/condition related angiogenic process or, alternatively, reduce or diminish the initiation of a naturally or disease/condition related angiogenic process.

“Angiogenesis mediated disorders,” as used herein, includes: (1) those disorders, diseases and/or unwanted conditions which are characterized by unwanted or elevated angiogenesis referred to herein collectively as “angiogenesis elevated disorders;” or (2) those disorders, diseases and/or unwanted conditions which are characterized by wanted or reduced angiogenesis referred to herein collectively as “angiogenesis reduced disorders.”

“KAPh,” is used herein in the broadest sense, and includes those proteins and nucleotides encoding the same or variants thereof. In one embodiment, KAPh is any protein comprising the catalytic domain of KAPh. In one embodiment, the region of KAPh containing the catalytic domain is characterized by SEQ ID NOS 3 and 4. In one embodiment, KAPh is the full-length sequence, that is, SEQ ID NOS 1 and 2. In one embodiment, KAPh is selected from the variants that are truncated, functional, or tagged form of the KAPh. In another embodiment, KAPh was cloned into a maltose binding protein vector to produce a fusion protein. The KAPh protein is cleaved away from the maltose binding protein using the TEV protease. In one embodiment, KAPh is an isolated KAPh. Non-limiting examples of KAPh are described in Reiko Kikuno, et al., DNA Research 6, 197-205 (1999); Huaiyan Chen et al., PNAS 99: 733-738 (2002); and Brad St. Crois, et al., Science 289, 1197-1202 (2002).

“VEGFR2,” is used herein in the broadest sense, and includes those proteins and nucleotides encoding the same or variants thereof. Potential “variants” of VEGFR2 receptors that might be used in the methods of the present invention include any truncated, functional, and or tagged form of the protein including the intracellular portion of the protein containing the kinase domain. The intracellular domain of VEGFR2 (KDR) starts at amino acid 790 as translated from the Genbank accession #AF063658. In one embodiment, VEGFR2 is any protein comprising the catalytic kinase domain of VEGFR2. In one embodiment, the VEGFR2 intracellular domain containing the catalytic domain is characterized by SEQ ID NO 7 and 8. In one embodiment, VEGFR2 is the full length sequence selected from SEQ ID NOS 5 and 6. In one embodiment, VEGFR2 is selected from the variants that are truncated, functional, or tagged form of the VEGFR2. In one embodiment, VEGFR2 is an isolated VEGFR2.

“Variants,” as used herein, means those proteins, or nucleotide sequences encoding the same (all used herein interchangeably unless otherwise indicated), that are substantially similar to those described by KAPh and VEGFR2, respectively. KAPh and VEGFR2 may be altered in various ways to yield a variant encompassed by the present invention including amino acid substitutions, deletions, truncations, insertions, and modifications. Methods for such manipulations are generally known in the art. For example, variants can be prepared by mutations in the nucleotide sequence. Methods for mutagenesis and nucleotide sequence alterations are well known in the art. See, for example, Kunkel, Proc. Natl. Acad. Sci. USA, 82, 488-492 (1985); Kunkel et al., Methods in Enzymol., 154, 367-382, (1987); U.S. Pat. No. 4,873,192; Walker and Gaastra, eds., Techniques in Molecular Biology, MacMillan Publishing Company, New York, (1983), and the references cited therein. In one embodiment of the variant, the substitution(s) of the protein is conservative in that it minimally disrupts the biochemical properties of the variant. Thus, where mutations are introduced to substitute amino acid residues, positively charged residues (H, K, and R) preferably are substituted with positively-charged residues; negatively-charged residues (D and E) preferably are substituted with negatively-charged residues; and neutral non-polar residues (A, F, I, L, M, P, V, and W) preferably are substituted with neutral non-polar residues. In another embodiment of the variant, the overall charge, structure or hydrophobic/hydrophilic properties of the protein can be altered without substantially adversely affecting the angiogenesis modulating capacity. In still another embodiment, the variant is an active fragment of a protein. In yet another embodiment of the variant, a protein is modified by acetylation, carboxylation, phosphorylation, glycosylation, ubiquitination, and labeling, whether accomplished by in vivo or in vitro enzymatic treatment of the protein or by the synthesis of the protein using modified amino acids. Non-limiting examples of modifications to amino acids include phosphorylation of tyrosine, serine, and threonine residues; methylation of lysine residues; acetylation of lysine residues; hydroxylation of proline and lysine residues; carboxylation of glutamic acid residues; glycosylation of serine, threonine, or asparagine residues; and ubiquitination of-lysine residues. The variant can also include other domains, such as epitope tags and His tags (e.g., the protein can be a fusion protein).

In yet another embodiment, peptide mimics of a KAPh or VEGFR2 are encompassed within the meaning of variant. As used herein, “mimic,” means an amino acid or an amino acid analog that has the same or similar functional characteristics of an amino acid. Thus, for example, an arginine analog can be a mimic of arginine if the analog contains a side chain having a positive charge at physiologic pH, as is characteristic of the guanidinium side chain reactive group of arginine. Examples of organic molecules that can be suitable mimics are listed at Table I of U.S. Pat. No. 5,807,819. Generally, a peptide variant, or nucleic acid sequence encoding the same, of the present invention will have at least 70%, generally, 80%, preferably up to 90%, more preferably 95%, even more preferably 97%, still even more preferably, and most preferably 99% sequence identity to its respective native amino acid sequence. Fusion proteins, or N-terminal, C-terminal or internal extensions, deletions, or insertions into the peptide sequence shall not be construed as affecting homology.

“Sequence Identity” or “Homology” at the amino acid or nucleotide sequence level is determined by BLAST (Basic Local Alignment Search Tool) analysis using the algorithm employed by the programs blastp, blastn, blastx, tblastn and tblastx, Altschul et al., Nucleic Acids Res. 25, 3389-3402 (1997) and Karlin et al. Proc. Natl. Acad. Sci., USA, 87, 2264-2268 (1990) which are tailored for sequence similarity searching. The approach used by the BLAST program is to first consider similar segments, with gaps (non-contiguous) and without gaps (contiguous), between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al. Nature Genetics, 6, 119-129 (1994). The search parameters for histograms, descriptions, alignments, (i.e., the statistical significance threshold for reporting matches against database sequences), cutoff, matrix and filter (low complexity) are at the default settings. The default scoring matrix used by blastp, blastx, tblastn, and tblastx is the BLOSUM62 matrix, Henikoff et al. Proc. Natl. Acad. Sci. USA 89, 10915-10919 (1992), recommended for query sequences over 85 nucleotides or amino acids in length.

For blastn, the scoring matrix is set by the ratios of M (i.e., the reward score for a pair of matching residues) to N (i.e., the penalty score for mismatching residues), wherein the default values for M and N are +5 and −4, respectively. Four blastn parameters were adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw-32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

“Isolated,” as used herein, refers to nucleic acid or amino acid sequences that are removed from their natural environment and are isolated or separated, and are at least about 60% free, preferably about 75% free, and most preferably about 90% free from other components with which they are naturally associated.

“Treatment,” as used herein, means, at a minimum, administration of an agent screened by the present invention that mitigates an angiogenesis mediated disorder in a mammalian subject, preferably in humans. Thus, the term “treatment” includes preventing an angiogenesis mediated disorder in a mammal, particularly when the mammal is predisposed to acquiring the disorder, but has not yet been diagnosed with the disorder; inhibiting the disorder; and/or alleviating or reversing the disorder. It is understood that the term “prevent” does not require that the disease state be completely thwarted. (See Webster's Ninth Collegiate Dictionary.) Rather, as used herein, the term preventing refers to the ability of the skilled artisan to identify a population that is susceptible to angiogenesis mediated disorders, such that administration of the agent may occur prior to the onset of the disease. The term does not imply that the disease state be completely avoided.

“Protein,” is used herein interchangeable with peptide and polypeptide.

I. KAPh

The invention is based on the discovery of a novel human KDR Associated Phosphatase (KAPh) as a modulator of angiogenesis mediated disorders. In turn, the discovery is based upon utilizing a yeast two-hybrid system to identify proteins that associate with the cytoplasmic domain of the KDR in an effort to discover novel intracellular interactions. In addition to isolating several previously reported interacting proteins, the investigators isolated and identified a novel cDNA as a KDR-interacting protein from screens of human umbilical vein endothelial cell (HUVEC) and human fetal brain cDNA libraries. Interestingly, KAPh exhibited high homology, particularly at the extreme carboxyl-terminus and in a region near the amino-terminus, with a focal adhesion molecule, tensin Davis S, et al., Science 1991; 252(5006):712-715; Lo S H, et al., J Biol Chem 1994; 269(35):22310-22319; Lo S H, et al., Bioessays 1994; 16(11):817-823; Haynie D T, Ponting C P, Protein Sci 1996; 5(12):2643-2646; and Chen H, et al., Biochem J 2000; 351 Pt 2:403-411. Like tensin, KAPh contains a C-terminal SH2 domain and an NH₂-terminal domain with very high homology to the catalytic region of protein tyrosine phosphatases/dual-specificity phosphatases (PTP/DSP). Closer inspection of the catalytic domain of KAPh, revealed a high degree of homology with the phosphatase domain of PTEN, a recently identified tumor suppressor protein Li J et al., Science 1997; 275(5308):1943-1947; Steck P A, et al., Nat Genet 1997; 15(4):356-362; Li D M et al., Cancer Res 1997; 57(11):2124-2129; and Li D M et al., Proc Natl Acad Sci U S A 1998; 95(26):15406-15411. When the KAPh phosphatase domain was tested for the ability to dephosphorylate a number of different substrates, reproducible phosphatase activity was observed with a fluorogenic substrate 6,8-difluoro-4-methylumbelliferone (DiFMUP). KAPh was highly expressed in both the vascular endothelium and surrounding smooth muscle of blood vessels in highly vascularized tissues, most prominently in heart, kidney, liver, lung, skeletal muscle, and brain. Based on its recruitment to the activated VEGFR2, its phosphatase activity, and its expression in the adult vasculature, the investigators believe that KAPh may be involved in cellular responses leading to angiogenesis and/or vascular maintenance.

One aspect of the invention provides for an isolated KAPh, and polynucleotide sequences encoding the same.

It will be appreciated by those skilled in the art that as a result of the degeneracy of the genetic code, a multitude of polynucleotide sequences encoding KAPh, some bearing minimal similarity to the polynucleotide sequences of any known and naturally occurring gene, may be produced. Thus, the invention contemplates each and every possible variation of polynucleotide sequence that could be made by selecting combinations based on possible codon choices. These combinations are made in accordance with the standard triplet genetic code as applied to the polynucleotide sequence of naturally occurring KAPh, and all such variations are to be considered as being specifically disclosed.

Although nucleotide sequences which encode KAPh and its variants are preferably capable of hybridizing to the nucleotide sequence of the naturally occurring KAPh under appropriately selected conditions of stringency (“conditions of stringency” as defined in Sambrook et al., Molecular Cloning: A Laboratory Manual, 2d edition, Cold Spring Harbor Press (1989)), it may be advantageous to produce nucleotide sequences encoding KAPh or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding KAPh and its derivatives without altering the encoded amino acid sequences include the production of RNA transcripts having more desirable properties, such as a greater half-life, than transcripts produced from the naturally occurring sequence.

The invention also encompasses production of DNA sequences which encode KAPh and variants thereof, entirely by synthetic chemistry. After production, the synthetic sequence may be inserted into any of the many available expression vectors and cell systems using reagents well known in the art. Moreover, synthetic chemistry may be used to introduce mutations into a sequence encoding KAPh or any variant thereof.

Also encompassed by the invention are polynucleotide sequences that are capable of hybridizing to the claimed polynucleotide sequences, and, in particular, to those shown in SEQ ID NO 1 under various conditions of stringency. (See, e.g., Wahl, G. M. and S. L. Berger (1987) Methods Enzymol. 152:399-407; Kimmel, A. R. (1987) Methods Enzymol. 152:507-511.) In one embodiment, an isolated polynucleotide which hybridizes to and which is at least 80%, preferable 90%, more preferably 95% complementary to a polynucleotide encoding a KAPh polypeptide or variant thereof.

Methods for DNA sequencing are well known and generally available in the art and may be used to practice any of the embodiments of the invention. The methods may employ such enzymes as the Klenow fragment of DNA polymerase I, Sequenase® (US Biochemical Corp., Cleveland, Ohio), Taq polymerase (Perkin Elmer), thermostable T7 polymerase (Amersham, Chicago, Ill.), or combinations of polymerases and proofreading exonucleases such as those found in the ELONGASE® Amplification System (GIBCO BRL, Gaithersburg, Md.). Preferably, the process is automated with machines such as the Hamilton Micro Lab 2200 (Hamilton, Reno, Nev.), Peltier Thermal Cycler (PTC200; M J Research, Watertown, Mass.) and the ABI Catalyst and 373 and 377 DNA Sequencers. (Perkin Elmer).

The nucleic acid sequences encoding KAPh may be extended utilizing a partial nucleotide sequence and employing various PCR-based methods known in the art to detect upstream sequences, such as promoters and regulatory elements. For example, one method which may be employed, restriction-site PCR, uses universal and nested primers to amplify unknown sequence from genomic DNA within a cloning vector. (See, e.g., Sarkar, G. (1993) PCR Methods Applic. 2:318-322.) Another method, inverse PCR, uses primers that extend in divergent directions to amplify unknown sequence from a circularized template. The template is derived from restriction fragments comprising a known genomic locus and surrounding sequences. (See, e.g., Triglia, T. et al. (1988) Nucleic Acids Res. 16:8186.) A third method, capture PCR, involves PCR amplification of DNA fragments adjacent to known sequences in human and yeast artificial chromosome DNA. (See, e.g., Lagerstrom, M. et al. (1991) PCR Methods Applic. 1:111-119.) In this method, multiple restriction enzyme digestions and ligations may be used to insert an engineered double-stranded sequence into a region of unknown sequence before performing PCR. Other methods which may be used to retrieve unknown sequences are known in the art. (See, e.g., Parker, J. D. et al. (1991) Nucleic Acids Res. 19:3055-306). Additionally, one may use PCR, nested primers, and PromoterFinder® libraries to walk genomic DNA (Clontech, Palo Alto, Calif.). This procedure avoids the need to screen libraries and is useful in finding intron/exon junctions. For all PCR-based methods, primers may be designed using commercially available software, such as OLIGO® 4.06 Primer Analysis software (National Biosciences Inc., Plymouth, Minn.) or another appropriate program, to be about 22 to 30 nucleotides in length, to have a GC content of about 50% or more, and to anneal to the template at temperatures of about 68 degrees Celsius to 72 degrees Celsius.

When screening for full-length cDNAs, it is preferable to use libraries that have been size-selected to include larger cDNAs. In addition, random-primed libraries, which often include sequences containing the 5′ regions of genes, are preferable for situations in which an oligo d(T) library does not yield a full-length cDNA. Genomic libraries may be useful for extension of sequence into 5′ non-transcribed regulatory regions.

Capillary electrophoresis systems that are commercially available may be used to analyze the size or confirm the nucleotide sequence of sequencing or PCR products. In particular, capillary sequencing may employ flowable polymers for electrophoretic separation, four different nucleotide-specific, laser-stimulated fluorescent dyes, and a charge coupled device camera for detection of the emitted wavelengths. Output/light intensity may be converted to electrical signal using appropriate software (e.g., Genotyper®] and Sequence Navigator®], Perkin Elmer), and the entire process from loading of samples to computer analysis and electronic data display may be computer controlled. Capillary electrophoresis is especially preferable for sequencing small DNA fragments which may be present in limited amounts in a particular sample.

In another embodiment of the invention, polynucleotide sequences or variants thereof which encode KAPh may be cloned in recombinant DNA molecules that direct expression of KAPh, or variants thereof, in appropriate host cells. As previously stated, due to the inherent degeneracy of the genetic code, other DNA sequences which encode substantially the same or a functionally equivalent amino acid sequence may be produced and used to express KAPh.

The nucleotide sequences of the present invention can be engineered using methods generally known in the art in order to alter KAPh-encoding sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

II. Methods of Screening an Agent Useful gor Modulating Angiogenesis.

The present invention is also based upon the discovery that KDR may represent a novel mechanism to regulate VEGF signaling and hence angiogenesis. In view of these surprising discoveries, KAPh may be used for screening agents useful in the treatment of angiogenesis mediated disorders in any of a variety of well-known drug screening techniques.

In one embodiment of the invention, KAPh or variants thereof (such variants including, but not limited to, KAPh catalytic or immunogenic fragments, or oligopeptides thereof) can be used for screening libraries of agents in any of a variety of drug screening techniques. The KAPh employed in such screening may be free in solution, affixed to a solid support, borne on a cell surface, or located intracellularly. The modulation of KAPh activity, KAPh expression, or KAPh and VEGFR2 association, by the agent being tested may be measured.

Another technique for agent screening provides for high throughput screening of agents having suitable binding affinity or association to the protein or nucleotide of interest. (See, e.g., Geysen, et al. (1984) PCT application WO84/03564.) In this method, large numbers of different test agents are synthesized on a solid substrate, such as plastic pins or some other surface. The test agents are reacted with KAPh, or fragments variants thereof, and washed. Bound KAPh is then detected by methods well known in the art. Purified KAPh can also be coated directly onto plates for use in the aforementioned drug screening techniques. Alternatively, non-neutralizing antibodies can be used to capture the protein and immobilize it on a solid support.

In yet another embodiment, one may use competitive drug screening assays in which neutralizing antibodies capable of binding KAPh specifically compete with a test agent for binding KAPh. In this manner, antibodies can be used to detect the presence of any peptide which shares one or more antigenic determinants with GPAP.

A. KAPh and VEGFR2

Isolated KAPh or VEGFR2 can be obtained by methods well known in the art. For example, KAPh or VEGFR2 may be synthesized using standard direct peptide synthesizing techniques (e.g., as summarized in Bodanszky, Principles of Peptide Synthesis, Springer-Verlag, Heidelberg: (1984)), such as via solid-phase synthesis (see, e.g., Merrifield, J. Am. Chem. Soc., 85, 2149-54 (1963) Roberge, J. Y. et al., Science 269: 202-204 (1995); Barany et al., Int. J. Peptide Protein Res., 30, 705-739 (1987); and U.S. Pat. No. 5,424,398). Of course, as genes for KAPh or VEGFR2 are known, disclosed herein, or can be deduced from the polypeptide sequences discussed herein. KAPh or VEGFR2 can be produced by standard recombinant methods. The proteins may be isolated or purified in a variety of ways known to those skilled in the art depending on what other components are present in the sample. Standard purification methods include electrophoretic, molecular, immunological, and chromatographic techniques, including ion exchange, hydrophobic, affinity, and reverse-phase HPLC chromatography, chromatofocussing, selective precipitation with such substances as ammonium sulfate; and others (see, e.g., Scopes, Protein Purification: Principles and Practice (1982); U.S. Pat. No. 4,673,641; and Sambrook et all, supra). For example, the target protein can be purified using a standard anti-target antibody column. Ultrafiltration and diafiltration techniques, in conjunction with protein concentration, are also useful.

For cell based assay in accordance with the present invention, cells comprising KAPh or VEGFR2 are well known in the art or can be modified to comprise KAPh and VEGFR2 by methods well known in the art. Suitable cells that naturally comprise KAPh and VEGFR2 include, but are not limited, liver, lung, skeletal muscle, and brain. Cell lines that comprise enhanced levels KAPh and VEGFR2 may be either purchased commercially or constructed. Well-known methods of providing cells with KAPh and VEGFR2 include incorporating an expression cassette, including a nucleic acid encoding KAPh and VEGFR2 to cells of interest. Standard transfection or transformation methods can be used to produce bacterial, mammalian, yeast or insect cell lines that express large quantities of protein, which are then purified using standard techniques (see, e.g., Colley et al., J. Biol. Chem. 264:17619-17622 (1989); Guide to Protein Purification, in Methods in Enzymology, vol. 82 (Deutscher ed., 1990)).

As discussed, coding sequences for KAPh or VEGFR2 are known, disclosed herein, and others can be deduced. Thus, KAPh and VEGFR2 expression cassettes typically employ coding sequences homologous to these known sequences, e.g., they will hybridize to at least a fragment of the known sequences under at least mild stringency conditions, more preferably under moderate stringency conditions, most preferably under high stringency conditions (employing the definitions of mild, moderate, and high stringency as set forth in Sambrook et al., Molecular Cloning: A Laboratory Manual, 2d edition, Cold Spring Harbor Press (1989)).

In addition to the KAPh or VEGFR2 coding sequence, an expression cassette includes a promoter, and, in the context of the present invention, the promoter must be able to drive the expression of the KAPh and VEGFR2 gene within the cells. Many viral promoters are appropriate for use in such an expression cassette (e.g., retroviral ITRs, LTRs, immediate early viral promoters (IEp) (such as herpesvirus IEp (e.g., ICP4-IEp and ICP0-IEp) and cytomegalovirus (CMV) IEp), and other viral promoters (e.g., late viral promoters, latency-active promoters (LAPs), Rous Sarcoma Virus (RSV) promoters, and Murine Leukemia Virus (MLV) promoters)). Other suitable promoters are eukaryotic promoters, such as enhancers (e.g., the rabbit beta-globin regulatory elements), constitutively active promoters (e.g., the beta-actin promoter, etc.), signal specific promoters (e.g., inducible and/or repressible promoters, such as a promoter responsive to TNF or RU486, the metallothionine promoter, etc.), and tumor-specific promoters.

Within the expression cassette, the KAPh or VEGFR2 gene and the promoter are operably linked such that the promoter is able to drive the expression of the KAPh or VEGFR2 gene. As long as this operable linkage is maintained, the expression cassette can include more than one gene, such as multiple genes separated by ribosome entry sites. Furthermore, the expression cassette can optionally include other elements, such as polyadenylation sequences, transcriptional regulatory elements (e.g., enhancers, silencers, etc.), or other sequences.

The expression cassette must be introduced into the cells in a manner suitable for them to express the KAPh or VEGFR2 gene contained therein. Any suitable vector can be so employed, many of which are known in the art. Examples of such vectors include naked DNA vectors (such as oligonucleotides or plasmids), viral vectors such as adeno-associated viral vectors, Bems et al., Ann. N.Y Acad. Sci., 772, 95-104 (1995), adenoviral vectors, Bain et al., Gene Therapy, 1, S68 (1994), herpesvirus vectors, Fink et al., Ann. Rev. Neurosci., 19, 265-87 (1996), packaged amplicons, Federoff et al., Proc. Nat. Acad. Sci. USA, 89, 1636-40 (1992), pappiloma virus vectors, picornavirus vectors, polyoma virus vectors, retroviral vectors, SV40 viral vectors, vaccinia virus vectors, and other vectors. A non-limiting example of a suitable vector is disclosed in U.S. Pat. Appl. 2001-0041679 A1.

The vector harboring the KAPh or VEGFR2 expression cassette is introduced into the cells by any means appropriate for the vector employed. Many such methods are well-known in the art (Sambrook et al., supra; see also Watson et al., Recombinant DNA, Chapter 12, 2d edition, Scientific American Books (1992)). Thus, plasmids are transferred by methods such as calcium phosphate precipitation, electroporation, liposome-mediated transfection, gene gun, microinjection, viral capsid-mediated transfer, polybrene-mediated transfer, protoplast fusion, etc. Viral vectors are best transferred into the cells by infecting them; however, the mode of infection can vary depending on the virus.

Successful expression of the KAPh or VEGFR2 gene can be assessed via standard molecular biological techniques (e.g., Northern hybridization, Western blotting, immunoprecipitation, enzyme immunoassay, etc.). For example, sequence-specific probes for KAPh or VEGFR2 can be generated which may be used in order to measure the expression levels of KAPh or VEGFR2 gene in a Northern blot hybridization assay. A single assay can be developed that could assess the levels of expression of various genes simultaneously. For example, antibodies can be generated against KAPh or VEGFR2 by techniques well known in the art (Harlow & Lane, Antibodies, A Laboratory Mannual (1998)). Both monoclonal and polyclonal antibodies are contemplated as a means for quantitation or for identifying the proteins. Alternatively, these proteins could be expressed in host cells as epitope-tagged proteins (e.g., histidine tag, myc tag, etc.). Antibodies are commercially available against these tagged portions and can selectively identify the proteins that contain the tag. Antibodies, thus developed may be used in Western blotting, ELISA and other immunoassays.

B. Measuring Activity of KAPh.

The activity of KAPh can be measured by methods well known in the art, Wang Y, Journal of Biological Chemistry, 267, 16696, 1992; Harder Kwet al., Biochemistry Journal, 298, 395, 1994; Itoh et al., Journal of Biological Chemistry, 267, 12356, 1992. In one embodiment, phosphatase activity measured. In one format, the phosphatase activity is measured using a fluorescent assay that generates a fluorescent signal when the substrate is acted upon by the enzyme. Other small molecule phosphatase substrates such as PNPP (para nitro phenyl phosphate) could also be used. These assay formats may be scaled-up for utilization in a high throughput screening assays using FRET (fluorescence resonance energy transfer) FP (Fluorescence polarization) or Malachite green assay. Another means of assaying for KAPh activity is to measure the loss of phosphorylation of its targets, e.g., VEGFR2 or receptor tyrosine kinase domain fusion proteins or synthetic peptides carrying phosphotyrosine residues mimicking the VEGFR2 phosphorylation sites or, alternatively based on the similarity of the KAPh and PTEN phosphatase domain, natural or synthetic phospholipid substrates. A phosphotyrosine western blot may be used to determine the decrease in phosphotyrosine content of a target protein in a suitable assay format Huang et al., Journal of Biological Chemistry, 274, 38183-38188, 1999.

C. Measuring Expression of KAPh.

The expression of KAPh can be measured by methods well known in the art. In general, host cells that contain the nucleic acid sequence encoding KAPh and that express KAPh may be identified by a variety of procedures known to those of skill in the art. These procedures include, but are not limited to, DNA-DNA or DNA-RNA hybridizations, PCR amplification, and protein bioassay or immunoassay techniques which include membrane, solution, or chip based technologies for the detection and/or quantification of nucleic acid or protein sequences.

Immunological methods for detecting and measuring the expression of KAPh using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on KAPh is preferred, but a competitive binding assay may be employed. These and other assays are well known in the art. (See, e.g., Hampton, R. et al. (1990) Serological Methods, a Laboratory Manual, APS Press, St Paul, Minn., Section IV; Coligan, J. E. et al. (1997 and periodic supplements) Current Protocols in Immunology, Greene Pub. Associates and Wiley-Interscience, New York, N.Y.; and Maddox, D. E. et al. (1983) J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides encoding KAPh include oligolabeling, nick translation, end-labeling, or PCR amplification using a labeled nucleotide. Alternatively, the sequences encoding KAPh, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Pharmacia & Upjohn (Kalamazoo, Mich.), Promega (Madison, Wis.), and U.S. Biochemical Corp. (Cleveland, Ohio). Suitable reporter molecules or labels which may be used for ease of detection include radionuclides, enzymes, fluorescent, chemiluminescent, or chromogenic agents, as well as substrates, cofactors, inhibitors, magnetic particles, and the like.

D. Measuring the Association of KAPh and VEGFR2.

The term “association,” as used in the phrase “association of KAPh and VEGFR2,” means KAPh and VEGFR2 interacting directly via a protein-protein interaction. This association may be measured in the presence or absence of an agent by those methods well known in the art.

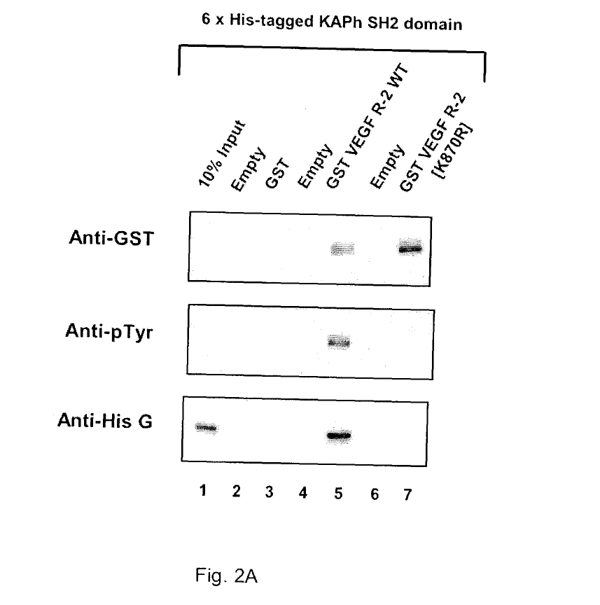

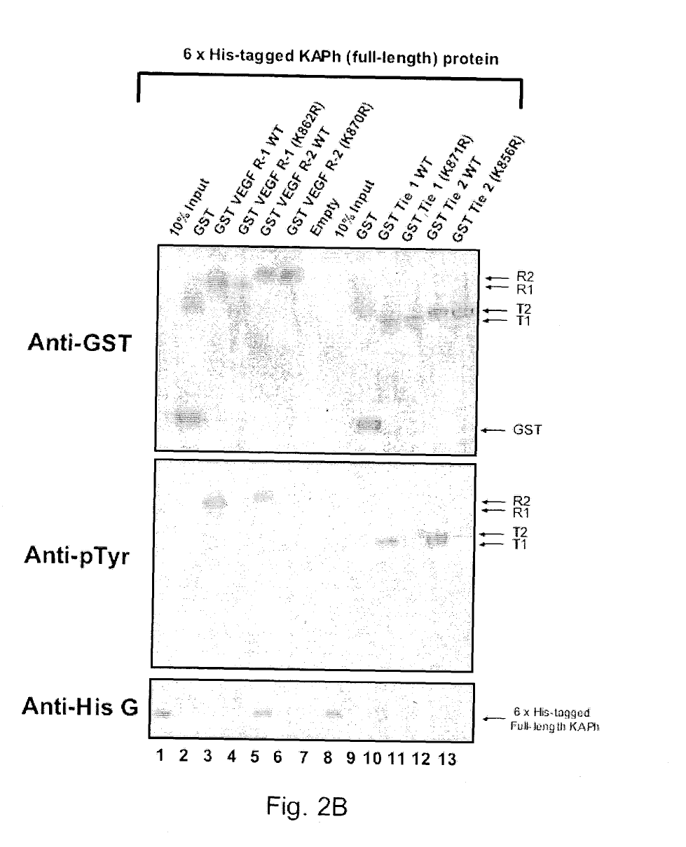

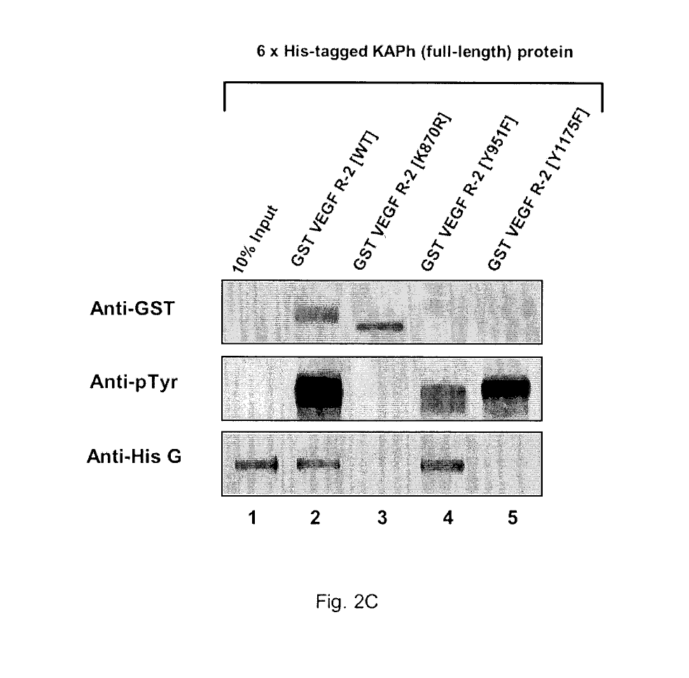

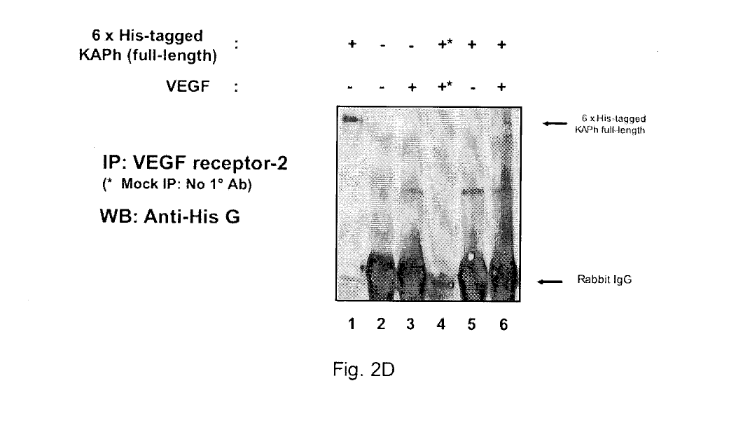

In one embodiment, association of KAPh and VEGFR2 is measured by co immunoprecipitation. For example, receptors are immunoprecipitated from cells that coexpress VEGFR2 and KAPh (either the endogenous proteins or by transfection). The immunoprecipitated receptors are then resolved by SDS-PAGE and associated proteins are detected by Western blot analysis (FIG. 2D). The KAPh associated with VEGFR2 is detected using anti-KAPh antibodies and the VEGFR2 is detected using commercial antibodies directed against the intracellular domain of VEGFR2. Conversely, KAPh immunoprecipitates could be Western Blotted for associated VEGFR2.

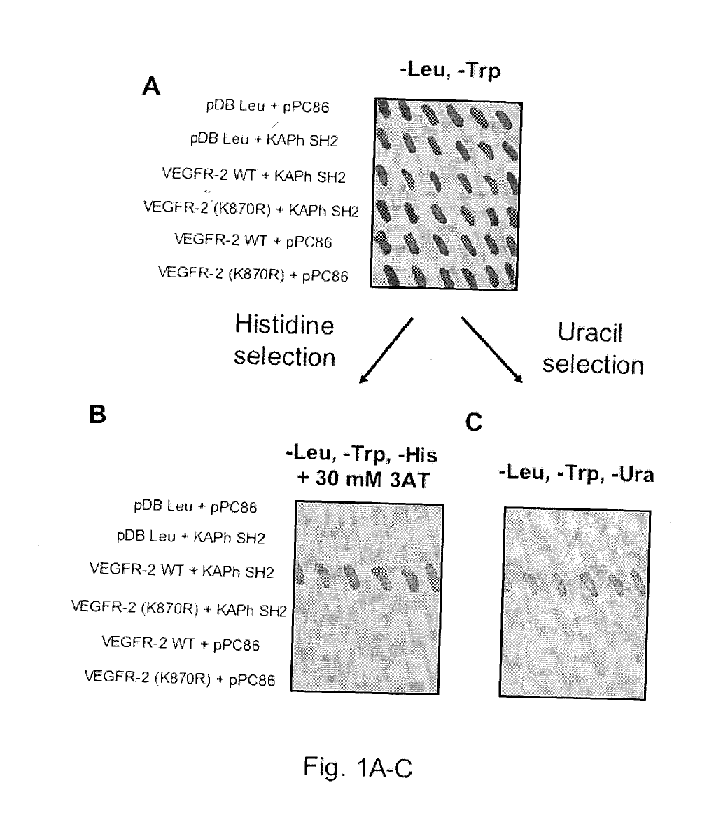

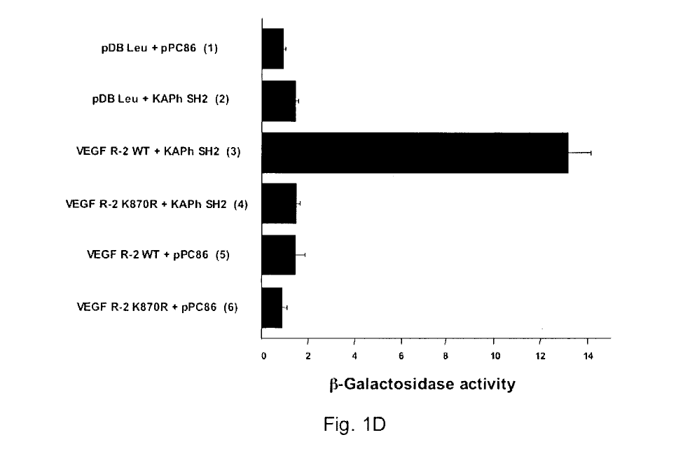

In another embodiment, a yeast 2-hybrid system is used (e.g., FIG. 1A-D).

In another embodiment, a pull-down assay is used wherein a tagged, recombinant protein (KAPh or VEGFR2) is immobilized and exposed to either cell lysates containing KAPh or VEGFR2 or the recombinant KAPh or VEGFR2 (e.g., FIG. 2A-C).

III. Agent

As used herein, the term “agent,” is used in the broadest sense, to include, without limitation: peptides, peptidomimetics, chemical compounds, nucleotides, antibodies, small molecules, vitamin derivatives, or carbohydrates. In one embodiment, the agent is an agonist. In another embodiment, the agent is an antagonist.

For example, an agent that may modulate KAPh gene expression is a polynucleotide. The polynucleotide may be an antisense, a triplex agent, or a ribozyme. For example, an anti sense may be directed to the structural gene region or to the promoter region of an KAPh gene.

In another example, an agent that may modulate KAPh translation is an antisense nucleic acid or ribozyme that could be used to bind to the KAPh mRNA or to cleave it. Antisense RNA or DNA molecules bind specifically with a targeted gene's mRNA message, interrupting the expression of that gene's protein product.

In one format, in the screening for an agent that modulates the expression of KAPh, the assay format is such that the cell lines that contain reporter gene fusions between the open reading frame defined by nucleotides encoding KAPh and/or the 5′ and/or 3′ regulatory elements and any assayable fusion partner may be prepared. Numerous assayable fusion partners are known and readily available including the firefly luciferase gene and the gene encoding chloramphenicol acetyltransferase, Alam et al. Anal. Biochem., 188, 245-254 (1990). Cell lines containing the reporter gene fusions are then exposed to the agent to be tested under appropriate conditions and time. Differential expression of the reporter gene between samples exposed to the agent and control samples identifies agents that modulate the expression of a nucleic acid encoding KAPh.

Additional assay formats may be used to monitor the ability of the agent to modulate the expression of a nucleic acid encoding KAPh. For instance, mRNA expression may be monitored directly by hybridization to the nucleic acids of encoding KAPh. Another way to evaluate KAPh expression levels is to use either quantitative or semi-quantitative PCR. Semi-quantitative PCR is performed by using a thermo-stable DNA polymerase and temperature cycling to “amplify” a portion of a given cDNA species using specific oligonuceotide primers as described in Sambrook et al., Molecular Cloning: A Laboratory Manual, 2d edition, Cold Spring Harbor Press (1989). An example of quantitative PCR is the use of TaqMan™ analysis developed and described by Applied Biosystems, (ABI). Cell lines are exposed to the agent to be tested under appropriate conditions and time and total RNA or mRNA is isolated by standard procedures such those disclosed in Sambrook et al. Molecular Cloning—A Laboratory Manual, Cold Spring Harbor Laboratory Press (1989). An example of a suitable probe is SEQ ID NO 7.

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementation that should exist between two nucleic acid strands in order to form a hybrid. Stringency should be chosen to maximize the difference in stability between the probe:target hybrid and probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al., Molecular Cloning—A Laboratory Manual, Cold Spring Harbor Laboratory Press, (1989) or Ausubel et al., Current Protocols in Molecular Biology, Greene Publishing Co. (1995).

Hybridization conditions are modified using known methods, such as those described by Sambrook et al. and Ausubel et al. as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a silicon chip or a porous glass wafer. The glass wafer can then be exposed to total cellular RNA or polyA RNA from a sample under conditions in which the affixed sequences will specifically hybridize. Such solid supports and hybridization methods are widely available, for example, those disclosed in WO 95/11755. By examining for the ability of a given probe to specifically hybridize to an RNA sample from an untreated cell population and from a cell population exposed to the agent, agents which up or down regulate the expression of a nucleic acid encoding HPTPbeta are identified.

Hybridization for qualitative and quantitative analysis of mRNA may also be carried out by using a RNase Protection Assay (i.e., RPA, see Ma et al., Methods 10, 273-238 (1996)). Briefly, an expression vehicle comprising cDNA encoding the gene product and a phage specific DNA dependent RNA polymerase promoter (e.g., T7, T3 or SP6 RNA polymerase) is linearized at the 3′ end of the cDNA molecule, downstream from the phage promoter, wherein such a linearized molecule is subsequently used as a template for synthesis of a labeled antisense transcript of the cDNA by in vitro transcription. The labeled transcript is then hybridized to a mixture of isolated RNA (i.e., total or fractionated mRNA) by incubation at 45° C. overnight in a buffer comprising 80% formamide, 40 mM Pipes (pH 6.4), 0.4 M NaCl and 1 mM EDTA. The resulting hybrids are then digested in a buffer comprising 40 ug/ml ribonuclease A and 2 ug/ml ribonuclease. After deactivation and extraction of extraneous proteins, the samples are loaded onto urea/polyacrylamide gels for analysis.

In another assay format, cells or cell lines are first identified which express the gene products of KAPh physiologically. Cell and/or cell lines so identified would be expected to comprise the necessary cellular-machinery such that the fidelity of modulation of the transcriptional apparatus is maintained with regard to exogenous contact of agent with appropriate surface transduction mechanisms and/or the cytosolic cascades. Further, such cells or cell lines would be transduced or transfected with an expression vehicle (e.g., a plasmid or viral vector) construct comprising an operable non-translated 5′-promoter containing end of the structural gene encoding the instant gene products fused to one or more antigenic fragments, which are peculiar to the instant gene products, wherein said fragments are under the transcriptional control of said promoter and are expressed as polypeptides whose molecular weight can be distinguished from the naturally occurring polypeptides or may further comprise an immunologically distinct tag or other detectable marker. Such a process is well known in the art (see Sambrook et al., Molecular Cloning—A Laboratory Manual, Cold Spring Harbor Laboratory Press (1989)).

Cells or cell lines transduced or transfected as outlined above are then contacted with agents under appropriate conditions; for example, the agent in a pharmaceutically acceptable excipient is contacted with cells in an aqueous physiological buffer such as phosphate buffered saline (PBS) at physiological pH, Eagles balanced salt solution (BSS) at physiological pH, PBS or BSS comprising serum or conditioned media comprising PBS or BSS and/or serum incubated at 37° C. Said conditions may be modulated as deemed necessary by one skilled in the art. Subsequent to contacting the cells with the agent, said cells will be disrupted and the polypeptides of the lysate are fractionated such that a polypeptide fraction is pooled and contacted with an antibody to be further processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the agent-contacted sample compared to the control will be used to distinguish the effectiveness of the agent.

In one format, the specific activity of KAPh is normalized to a standard unit, between a cell population that has been exposed to the agent to be tested and compared to an un-exposed control cell population. Cell lines or populations are exposed to the agent to be tested under appropriate conditions and time. Cellular lysates may be prepared from the exposed cell line or population and a control, unexposed cell line or population. The cellular lysates are then analyzed with a probe.

Antibody probes can be prepared by immunizing suitable mammalian hosts utilizing appropriate immunization protocols using the proteins of the invention or antigen-containing fragments thereof. While the polyclonal antisera produced in this way may be satisfactory for some applications, for pharmaceutical compositions, use of monoclonal preparations is preferred. Immortalized cell lines which secrete the desired monoclonal antibodies may be prepared using standard methods, (see e.g., Kohler & Milstein, Biotechnology, 24, 524-526 (1992) or modifications which affect immortalization of lymphocytes or spleen cells, as is generally known.

Fragments of the monoclonal antibodies or the polyclonal antisera that contain the immunologically significant portion can be used as antagonists, as well as the intact antibodies. Use of immunologically reactive fragments, such as Fab or Fab′ fragments, is often preferable, especially in a therapeutic context, as these fragments are generally less immunogenic than the whole immunoglobulin.

The antibodies or fragments may also be produced, using current technology, by recombinant means. Antibody regions that bind specifically to the desired regions of the protein can also be produced in the context of chimeras with multiple species origin, for instance, humanized antibodies. The antibody can therefore be a humanized antibody or human antibody, as described in U.S. Pat. No. 5,585,089 or Riechmann et al., Nature 332, 323-327 (1988).

One class of agents that may modulate KAPh activity includes peptide mimetics that mimic the three-dimensional structure of a KAPh protein. Such peptide mimetics may have significant advantages over naturally occurring peptides, including, for example: more economical production, greater chemical stability, enhanced pharmacological properties (half-life, absorption, potency, efficacy, etc.), altered specificity (e.g., a broad-spectrum of biological activities), reduced antigenicity and others.

In one form, mimetics are peptide-containing molecules that mimic elements of protein secondary structure. The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule.

In another form, peptide analogs are commonly used in the pharmaceutical industry as non-peptide drugs with properties analogous to those of the template peptide. These types of non-peptide compounds are also referred to as peptide mimetics or peptidomimetics, Fauchere, Adv. Drug Res., 15, 29-69 (1986); Veber & Freidinger, Trends Neurosci., 8, 392-396 (1985); Evans et al., J. Med. Chem., 30, 1229-1239 (1987) which are incorporated herein by reference and are usually developed with the aid of computerized molecular modeling.

Peptide mimetics that are structurally similar to therapeutically useful peptides may be used to produce an equivalent therapeutic or prophylactic effect. Generally, peptide mimetics are structurally similar to a paradigm polypeptide (i.e., a polypeptide that has a biochemical property or pharmacological activity), but have one or more peptide linkages optionally replaced by a linkage by methods known in the art.

Labeling of peptide mimetics usually involves covalent attachment of one or more labels, directly or through a spacer (e.g., an amide group), to non-interfering positions on the peptide mimetic that are predicted by quantitative structure-activity data and molecular modeling. Such non-interfering positions generally are positions that do not form direct contacts with the macromolecules to which the peptide mimetic binds to produce the therapeutic effect. Derivitization (e.g., labeling) of peptide mimetics should not substantially interfere with the desired biological or pharmacological activity of the peptide mimetic.

The use of peptide mimetics can be enhanced through the use of combinatorial chemistry to create drug libraries. The design of peptide mimetics can be aided by identifying amino acid mutations that increase or decrease binding of the protein to its binding partners. Approaches that can be used include the yeast two hybrid method (see Chien et al., Proc. Natl. Acad. Sci., USA, 88, 9578-9582 (1991)) and using the phage display method. The two hybrid method detects protein-protein interactions in yeast, Fields et al., Nature, 340, 245-246 (1989). The phage display method detects the interaction between an immobilized protein and a protein that is expressed on the surface of phages such as lambda and M13, Amberg et al., Strategies, 6, 2-4 (1993); Hogrefe et al., Gene, 128, 119-126 (1993). These methods allow positive and negative selection for protein-protein interactions and the identification of the sequences that determine these interactions.

IV. Pharmaceutically Compositions

One aspect of the invention provides for a pharmaceutical composition comprising: (a) a safe and effective amount of KAPh or an agent modulating KAPh; and (b) a pharmaceutically-acceptable carrier. KAPh and said agents are formulated by standard pharmaceutical formulation techniques such as those disclosed in Remington's Pharmaceutical Sciences, Mack Publishing Company, Easton, Pa., latest edition.

A “safe and effective amount” means an amount of KAPh, or agent modulating KAPh, sufficient to significantly induce a positive modification in the condition to be treated, but low enough to avoid serious side effects (such as toxicity, irritation, or allergic response) in an animal, preferably a mammal, more preferably a human subject, in need thereof, commensurate with a reasonable benefit/risk ratio when used in the manner of this invention. The specific “safe and effective amount” will, obviously, vary with such factors as the particular condition being treated, the physical condition of the subject, the duration of treatment, the nature of concurrent therapy (if any), the specific dosage form to be used, the carrier employed, the solubility of the peptide therein, and the dosage regimen desired for the composition. One skilled in the art may use the following teachings to determine a “safe and effective amount” in accordance with the present invention. Spilker B., Guide to Clinical Studies and Developing Protocols, Raven Press Books, Ltd., New York, 1984, pp. 7-13, 54-60; Spilker B., Guide to Clinical Trials, Raven Press, Ltd., New York, 1991, pp. 93-101; Craig C., and R. Stitzel, eds., Modern Pharmacology, 2d ed., Little, Brown and Co., Boston, 1986, pp. 127-33; T. Speight, ed., Avery's Drug Treatment: Principles and Practice of Clinical Pharmacology and Therapeutics, 3d ed., Williams and Wilkins, Baltimore, 1987, pp. 50-56; R. Tallarida, R. Raffa and P. McGonigle, Principles in General Pharmacology, Springer-Verlag, New York, 1988, pp. 18-20.

In addition to the subject KAPh or agent, the compositions of the subject invention contain a pharmaceutically acceptable carrier. The term “pharmaceutically-acceptable carrier,” as used herein, means one or more compatible solid or liquid filler diluents or encapsulating substances which are suitable for administration to an animal, preferably a mammal, more preferably a human. The term “compatible”, as used herein, means that the components of the composition are capable of being commingled with the subject peptide, and with each other, in a manner such that there is no interaction that would substantially reduce the pharmaceutical efficacy of the composition under ordinary use situations. Pharmaceutically-acceptable carriers must, of course, be of sufficiently high purity and sufficiently low toxicity to render them suitable for administration to the animal, preferably a mammal, more preferably a human being treated.

Some examples of substances which can serve as pharmaceutically-acceptable carriers or components thereof are: sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the Tweens®; wetting agents, such sodium lauryl sulfate; coloring agents; flavoring agents; tableting agents, stabilizers; antioxidants; preservatives; pyrogen-free water; isotonic saline; and phosphate buffer solutions.

The choice of a pharmaceutically-acceptable carrier to be used in conjunction with the subject KAPh or agent is basically determined by the way the protein is to be administered.

If the subject KAPh or agent is to be injected, the preferred pharmaceutically-acceptable carrier is sterile, physiological saline, with a blood-compatible colloidal suspending agent, the pH of which has been adjusted to about 7.4.

In particular, pharmaceutically-acceptable carriers for systemic administration include sugars, starches, cellulose and its derivatives, malt, gelatin, talc, calcium sulfate, vegetable oils, synthetic oils, polyols, alginic acid, phosphate buffer solutions, emulsifiers, isotonic saline, and pyrogen-free water. Preferred carriers for parenteral administration include propylene glycol, ethyl oleate, pyrrolidone, ethanol, and sesame oil. Preferably, the pharmaceutically-acceptable carrier, in compositions for parenteral administration, comprises at least about 90% by weight of the total composition.

The compositions of this invention are preferably provided in unit dosage form. As used herein, a “unit dosage form” is a composition of this invention containing an amount of a KAPh or agent that is suitable for administration to an animal, preferably a mammal, more preferably a human subject, in a single dose, according to good medical practice. These compositions preferably contain from about 0.1 mg (milligrams) to about 1000 mg, more preferably from about 0.5 mg to about 500 mg, more preferably from about 1 mg to about 30 mg, of a KAPh or agent of the invention.

The compositions of this invention may be in any of a variety of forms, suitable, for example, for oral, rectal, topical, nasal, ocular or parenteral administration. Depending upon the particular route of administration desired, a variety of pharmaceutically-acceptable carriers well-known in the art may be used. These include solid or liquid fillers, diluents, hydrotropes, surface-active agents, and encapsulating substances. Optional pharmaceutically-active materials may be included, which do not substantially interfere with the therapeutic activity of the subject KAPh or agent. The amount of carrier employed in conjunction with the KAPh or agent is sufficient to provide a practical quantity of material for administration per unit dose of the KAPh or agent. Techniques and compositions for making dosage forms useful in the methods of this invention are described in the following references,: Modern Pharmaceutics, Chapters 9 and 10 (Banker & Rhodes, editors, 1979); Lieberman et al., Pharmaceutical Dosage Forms: Tablets (1981); and Ansel, Introduction to Pharmaceutical Dosage Forms 2d Edition (1976).

Various oral dosage forms can be used, including such solid forms as tablets, capsules, granules and bulk powders. These oral forms comprise a safe and effective amount, usually at least about 5%, and preferably from about 25% to about 50%, of the KAPh or agent. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, and containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration are well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmelose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical for the purposes of the subject invention, and can be readily made by a person skilled in the art. In general, the formulation will include the peptide, and inert ingredients which allow for protection against the stomach environment, and release of the biologically active material in the intestine.

KAPh or protein agents may be chemically modified so that oral delivery of the derivative is efficacious. Generally, the chemical modification contemplated is the attachment of at least one moiety to the protein molecule itself, where said moiety permits (a) inhibition of proteolysis; and (b) uptake into the blood stream from the stomach or intestine. Also desired is the increase in overall stability of the protein and increase in circulation time in the body. Examples of such moieties include: polyethylene glycol, copolymers of ethylene glycol and propylene glycol, carboxymethyl cellulose, dextran, polyvinyl alcohol, polyvinyl pyrrolidone and polyproline. Newmark et al., J. Appl. Biochem., 4:185-189 (1982). Other polymers that could be used are poly-1,3-dioxolane and poly-1,3,6-tioxocane. Preferred for pharmaceutical usage, as indicated above, are polyethylene glycol moieties.

The location of release may be the stomach, the small intestine (the duodenum, the jejunem, or the ileum), or the large intestine. One skilled in the art has available formulations which will not dissolve in the stomach, yet will release the material in the duodenum or elsewhere in the intestine. Preferably, the release will avoid the deleterious effects of the stomach environment, either by protection of the peptide (or variant) or by release of the biologically active material beyond the stomach environment, such as in the intestine.

To ensure full gastric resistance, a coating impermeable to at least pH 5.0 is preferred. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, Avicel® RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Compositions of the subject invention may optionally include other active agents. Non-limiting examples of active agents are listed in WO 99/15210.

Other compositions useful for attaining systemic delivery of the subject compounds include sublingual, buccal, suppository, nasal and pulmonary dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

The compositions of this invention can also be administered topically to a subject, e.g., by the direct laying on or spreading of the composition on the epidermal or epithelial tissue of the subject, or transdermally via a “patch.” Such compositions include, for example, lotions, creams, solutions, gels and solids. These topical compositions preferably comprise a safe and effective amount, usually at least about 0.1%, and preferably from about 1% to about 5%, of the peptide. Suitable carriers for topical administration preferably remain in place on the skin as a continuous film, and resist being removed by perspiration or immersion in water. Generally, the carrier is organic in nature and capable of having dispersed or dissolved therein the peptide. The carrier may include pharmaceutically-acceptable emollients, emulsifiers, thickening agents, solvents and the like.

V. Treatment of Angiogenesis Mediated Disorder

KAPh or a KAPh modulating agent may be used in a method for the treatment of an angiogenesis mediated disorder.

A. Treatment of Angiogenesis Elevated Disorder.

In one aspect in the method for the treatment of an angiogenesis mediated disorder, a KAPh or KAPh modulating agent may be used in a method for the treatment of an “angiogenesis elevated disorder.” As used herein, an “angiogenesis elevated disorder” is one that involves unwanted or elevated angiogenesis in the biological manifestation of the disease, disorder, and/or condition; in the biological cascade leading to the disorder; or as a symptom of the disorder. This “involvement” of angiogenesis in an angiogenesis elevated disorder includes, but is not limited to, the following: (1) The unwanted or elevated angiogenesis as a “cause” of the disorder or biological manifestation, whether the level of angiogenesis is elevated genetically, by infection, by autoimmunity, trauma, biomechanical causes, lifestyle, or by some other causes. (2) The angiogenesis as part of the observable manifestation of the disease or disorder. That is, the disease or disorder is measurable in terms of the increased angiogenesis. From a clinical standpoint, unwanted or elevated angiogenesis indicate the disease, however, angiogenesis need not be the “hallmark” of the disease or disorder. (3) The unwanted or elevated angiogenesis is part of the biochemical or cellular cascade that results in the disease or disorder. In this respect, inhibition of angiogenesis interrupts the cascade, and thus controls the disease. Non-limiting examples of angiogenesis reduced disorders that may be treated by the present invention are herein described below.

A KAPh or KAPh modulating agent may be used to treat diseases associated with retinal/choroidal neovascularization that include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy, whether or not associated with diabetes.

A KAPh or KAPh modulating agent can treat diseases associated with chronic inflammation. Diseases with symptoms of chronic inflammation include inflammatory bowel diseases such as Crohn's disease and ulcerative colitis, psoriasis, sarcoidosis and rheumatoid arthritis. Angiogenesis is a key element that these chronic inflammatory diseases have in common. The chronic inflammation depends on continuous formation of capillary sprouts to maintain an influx of inflammatory cells. The influx and presence of the inflammatory cells produce granulomas and thus, maintain the chronic inflammatory state. Inhibition of angiogenesis by the compositions and methods of the present invention would prevent the formation of the granulomas and alleviate the disease.

A KAPh or KAPh modulating agent may used to treat patients with inflammatory bowel diseases such as Crohn's disease and ulcerative colitis. Both Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells. Prevention of angiogenesis by the HPTPbetas of the present invention inhibits the formation of the sprouts and prevents the formation of granulomas. Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea.

The inflammatory bowel diseases also show extraintestinal manifestations such as skin lesions. Such lesions are characterized by inflammation and angiogenesis and can occur at many sites other than the gastrointestinal tract. The agents may be capable of treating these lesions by preventing the angiogenesis, thus reducing the influx of inflammatory cells and the lesion formation.

Sarcoidosis is another chronic inflammatory disease that is characterized as a multisystem granulomatous disorder. The granulomas of this disease may form anywhere in the body and thus the symptoms depend on the site of the granulomas and whether the disease active. The granulomas are created by the angiogenic capillary sprouts providing a constant supply of inflammatory cells.

A KAPh or KAPh modulating agent can also treat the chronic inflammatory conditions associated with psoriasis. Psoriasis, a skin disease, is another chronic and recurrent disease that is characterized by papules and plaques of various sizes. Prevention of the formation of the new blood vessels necessary to maintain the characteristic lesions leads to relief from the symptoms.

Another disease that may be treated according to the present invention, is rheumatoid arthritis. Rheumatoid arthritis is a chronic inflammatory disease characterized by nonspecific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Other diseases that can be treated according to the present invention are hemangiomas, Osler-Weber-Rendu disease, or hereditary hemorrhagic telangiectasia, solid or blood borne tumors and acquired immune deficiency syndrome.

B. Treatment of an Angiogenesis Reduced Disorder.

In one aspect in the method for the treatment of an angiogenesis mediated disorder, a KAPh or KAPh modulating agent may be used in a method for the treatment of an “angiogenesis reduced disorder.” As used herein, an “angiogenesis reduced disorder” is one that involves stimulated angiogenesis to treat a disease, disorder, or condition. The disorder is one characterized by tissue that is suffering from or be at risk of suffering from ischemic damage, infection, and/or poor healing, which results when the tissue is deprived of an adequate supply of oxygenated blood due to inadequate circulation. As used herein, “tissue” is used in the broadest sense, to include, but not limited to, the following: cardiac tissue, such as myocardium and cardiac ventricles; erectile tissue; skeletal muscle; neurological tissue, such as from the cerebellum; internal organs, such as the brain, heart, pancreas, liver, spleen, and lung; or generalized area of the body such as entire limbs, a foot, or distal appendages such as fingers or toes. This inadequate blood supply to tissue includes the following: (1) The inadequate blood supply as a “cause” of the disorder or the biological manifestation thereof, whether the level of blood supply is reduced genetically, by infection, by autoimmunity, trauma, surgery, biomechanical causes, lifestyle, or by some other causes. (2) The inadequate blood supply as part of the observable manifestation of the disorder. That is, the disorder is measurable in terms of the inadequate blood supply. From a clinical standpoint, inadequate blood supply indicates the disease, however, inadequate blood supply need not be the “hallmark” of the disorder. (3) The inadequate blood supply is part of the biochemical or cellular cascade that results in the disorder. In this respect, stimulation of angiogenesis interrupts the cascade, and thus controls the disorder. Non-limiting examples of angiogenesis reduced disorders that may be treated by the present invention are herein described below.

1. Method of Vascularizing Ischemic Tissue

In one aspect in the method for the treatment of an angiogenesis reduced disorders, a KAPh or KAPh modulating agent may be used in a method of vascularizing ischemic tissue. As used herein, “ischemic tissue,” means tissue that is deprived of adequate blood flow. Examples of ischemic tissue include, but are not limited to, tissue that lack adequate blood supply resulting from mycocardial and cerebral infarctions, mesenteric or limb ischemia, or the result of a vascular occlusion or stenosis. In one example, the interruption of the supply of oxygenated blood may be caused by a vascular occlusion. Such vascular occlusion can be caused by arteriosclerosis, trauma, surgical procedures, disease, and/or other etiologies. There are many ways to determine if a tissue is at risk of suffering ischemic damage from undesirable vascular occlusion. Such methods are well known to physicians who treat such conditions. For example, in myocardial disease these methods include a variety of imaging techniques (e.g., radiotracer methodologies, x-ray, and MRI) and physiological tests. Therefore, induction of angiogenesis in tissue affected by or at risk of being affected by a vascular occlusion is an effective means of preventing and/or attenuating ischemia in such tissue. Thus, the treatment of skeletal muscle and myocardial ischemia, stroke, coronary artery disease, peripheral vascular disease are fully contemplated.

Any person skilled in the art of using standard techniques can measure the vascularization of tissue. Non-limiting examples of measuring vascularization in a subject include: SPECT (single photon emission computed tomography); PET (positron emission tomography); MRI (magnetic resonance imaging); and combination thereof, by measuring blood flow to tissue before and after treatment. Angiography can be used as an assessment of macroscopic vascularity. Histologic evaluation can be used to quantify vascularity at the small vessel level. These and other techniques are discussed in Simons, et al., “Clinical trials in coronary angiogenesis,” Circulation, 102, 73-86 (2000).

2. Method of Repairing Tissue

In one aspect in the method for the treatment of an angiogenesis reduced disorder, a KAPh or KAPh modulating agent may be used in a method of repairing tissue. As used herein, “repairing tissue” means promoting tissue repair, regeneration, growth, and/or maintenance including, but not limited to, wound repair or tissue engineering. One skilled in the art readily appreciates that new blood vessel formation is required for tissue repair. In turn, tissue may be damaged by, including, but not limited to, traumatic injuries or conditions including arthritis, osteoporosis and other skeletal disorders, and burns. Tissue may also be damaged by results from injuries due to surgical procedures, irradiation, laceration, toxic chemicals, viral infection bacterial infection or burns. Tissue in need of repair also includes non-healing wounds. Non-limiting examples of non-healing wounds include: non-healing skin ulcers resulting from diabetic pathology; or fractures that do not heal readily.

A KAPh or agent may also be used in a method to aid in tissue repair in the context of guided tissue regeneration (GTR) procedures. Such procedures are currently used by those skilled in the medical arts to accelerate wound healing following invasive surgical procedures. KAPh or agent may be used in a method of promoting tissue repair characterized by enhanced tissue growth during the process of tissue engineering. As used herein, “tissue engineering” is defined as the creation, design, and fabrication of biological prosthetic devices, in combination with synthetic or natural materials, for the augmentation or replacement of body tissues and organs. Thus, the present method can be used to augment the design and growth of human tissues outside the body for later implantation in the repair or replacement of diseased tissues. For example, KAPh or agent may be useful in promoting the growth of skin graft replacements that are used as a therapy in the treatment of burns.

In another aspect of tissue engineering, KAPh or agent of the present invention may be included in cell-containing or cell-free devices that induce the regeneration of functional human tissues when implanted at a site that requires regeneration. As previously discussed, biomaterial-guided tissue regeneration can be used to promote bone regrowth in, for example, periodontal disease. Thus, a KAPh or agent may be used to promote the growth of reconstituted tissues assembled into three-dimensional configurations at the site of a wound or other tissue in need of such repair.

In another aspect of tissue engineering, KAPh or agent can be included in external or internal devices containing human tissues designed to replace the function of diseased internal tissues. This approach involves isolating cells from the body, placing them on or within structural matrices, and implanting the new system inside the body or using the system outside the body. The method of the invention can be included in such matrices to promote the growth of tissues contained in the matrices. For example, a KAPh or agent can be included in a cell-lined vascular graft to promote the growth of the cells contained in the graft. It is envisioned that the method of the invention can be used to augment tissue repair, regeneration and engineering in products such as cartilage and bone, central nervous system tissues, muscle, liver, and pancreatic islet (insulin-producing) cells.

VI. Diagnostic or Prognostic Methods

Expression of KAPh may be used as a diagnostic marker for the prediction or identification of an angiogenesis mediated disorder. For example, a cell or tissue sample may be assayed for the expression levels of a KAPh by any of the methods described herein and compared to the expression level found in normal healthy tissue. Such methods may be used to diagnose or identify angiogenesis mediated disorders.

Expression of KAPh may also be used as a marker for the monitoring the progression of an angiogenesis mediated disorder. Expression or activity of the KAPh may also used to track or predict the progress or efficacy of a treatment regime in a patient. For instance, a patient's progress or response to a given drug may be monitored by measuring gene expression of a KAPh of the invention in a cell or tissue sample after treatment or administration of the drug. The expression of KAPh in the post-treatment sample may then be compared to gene expression from the same patient before treatment.

### EXAMPLE

Experimental Procedures

Yeast Two-Hybrid Screening

All two-hybrid plasmid constructs used the pDBLeu and pPC86 yeast expression vectors as a part of the ProQuest Two-Hybrid System (Life Technologies, Rockville, Md.). A cDNA encoding the intracellular domain of human VEGFR2 was generated by PCR from a human VEGFR2/pJFE14 construct, obtained from Regeneron Pharmaceuticals (Tarrytown, N.Y.), and subcloned into the yeast bait vector downstream of the GAL4 DNA-binding domain. A human umbilical vein endothelial cell (HUVEC) cDNA library (in pPC86) was co-transformed with pDBLeu-hVEGFR2 into the MaV203 strain of yeast. Proteins that interacted with the intracellular domain of human VEGFR2 were selected and tested for specificity according to the manufacturer's instructions.

Oligonucleotide-Directed Mutagensis

The generation of kinase inactive mutants of human VEGFR-1, VEGFR2, Tie1, and Tie2 were performed using a QuickChange site-directed mutagenesis kit from Stratagene (La Jolla, Calif.). Mutation of the ATP binding site (K→R) in the intracellular kinase domain of these proteins rendered them catalytically inactive. Primer pairs containing the desired mutations were designed and primer extensions were performed using the reagents and protocol provided by the manufacturer. The entire cDNA insert for each construct was sequenced to confirm the presence of the desired mutation and absence of any aberrant mutations.

Isolation of Human KAPh cDNA

A Rapid-Screen arrayed human heart cDNA library was purchased from OriGene Technologies, Inc. (Rockville, Md.). A master plate and subplates of this library were screened by PCR as recommended by the manufacturer until a single plasmid was identified. The entire KAPh ORF was sequenced three times to confirm the precise nucleotide sequence.

Expression and Purification of Recombinant Proteins

Recombinant VEGFR-1 kinase (wild-type and K862R mutant), VEGFR2 kinase (wild-type and K870R mutant), Tie1 kinase (wild-type and K871R mutant), and Tie2 kinase (wild-type and K856R mutant) were expressed as glutathione S-transferase (GST) fusion proteins in Sf9 cells using a baculovirus expression system. Briefly, the intracellular domain of each receptor tyrosine kinase was generated as an in-frame GST fusion using the pAcGHLT baculovirus transfer vector (Pharmingen, San Diego, Calif.). Each baculovirus transfer vector construct was cotransfected with BaculoGold (Pharmingen, San Diego, Calif.) linearized AcNPV baculovirus DNA into Sf9 cells for the generation of recombinant baculoviruses. Five days post-transfection, recombinant baculoviruses were harvested and amplified by re-infection of Sf9 cells. Each amplified baculovirus was then used to infect Sf9 cells for the expression of the recombinant kinases. Seventy-two hours after addition of baculovirus, infected Sf9 cells were lysed in 1% trition lysis buffer (20 mM Tris-Cl, pH 8.0, 137 mM NaCl, 10% glycerol, 1% triton X-100, 2 mM EDTA) containing the protease inhibitors phenylmethylsulfonyl fluoride, aprotinin, pepstatin A, and leupeptin. The recombinant GST fusion proteins were purified from the lysates by glutathione sepharose chromatography.

Association Assay

Baculovirus expressed GST fusions of wild-type and kinase inactive mutants of VEGFR-1, VEGFR2, Tie1, and Tie2 were immobilized on glutathione-sepharose beads, washed three times with 1% triton lysis buffer, twice with kinase buffer (20 mM Tris-Cl, pH 8.0, 100 mM NaCl, 12 mM MgCl₂, 1 mM DTT), and autophosphorylated in vitro in the presence of 1 mM ATP. The immobilized, autophosphorylated kinases were then incubated with HEK 293 cell lysates which overexpressed either the KAPh SH2 domain or KAPh full-length protein. After incubation with cell lysates, the protein complexes were washed three times with 0.1% triton lysis buffer and eluted with Laemmli buffer. KAPh protein that associated with the recombinant kinases was analyzed by Western blot using a chemiluminescence detection system (Amersham Pharmacia, Piscataway, N.J.).

Antibody Production

The VEGFR2 rabbit polyclonal antibody (R2.2) (Whitaker G B, et al., J Biol Chem 2001; 276(27):25520-25531) was raised against the purified peptide sequence Ac-SKRKSRPVSVKTFEDIPLEEPC-amide (amino acids 1225-1246 of SEQ ID NO 6), unique to the carboxyl-terminal domain of VEGFR2, and affinity purified by QCB, a division of BioSource International (Hopkinton, Mass.). The KAPh rabbit polyclonal antibody (62507 a.p.) was raised using purified recombinant human KAPh (amino acids 1054-1386 SEQ ID NO 2) as immunogen. The antibody was subsequently purified by QCB using an affinity column of the protein immunogen. The KAPh rabbit polyclonal antibody (62507 a.p.) recognizes both the KAPh SH2 domain and the full-length KAPh protein (data not shown).

Cell Culture, Transient Transfection, and Stable Cells

Human umbilical vein endothelial cells (HUVEC) obtained from Clonetics (Walkersville, Md.) were cultured in endothelial growth medium (Clonetics) and were used up to passage 3. HEK 293 cells obtained from the American Type Culture Collection (Manasses, Va.) were cultured in cell growth medium (Dulbecco's modified Eagle's medium supplemented with 10% fetal bovine serum, 1% L-glutamine, 1% nonessential amino acids, and 1% antimycotics). For stable expression, hVEGFR2 was subcloned into pLNCX (Clontech, Palo Alto, Calif.), transiently transfected into HEK 293 cells, and selected in the presence of Geneticin. The HEK 293 VEGFR2 clonal cell line was transiently transfected with either the SH2 domain or full-length KAPh in the mammalian expression vector pcDNA4/HisMax (Invitrogen, Carlsbad, Calif.). All constructs were transfected using LipofectAMINE PLUS (Life Technologies, Rockville, Md.) in serum-free DMEM according to the manufacturer's instructions.

Immunoprecipitation and Western Blotting

HEK 293 cells that stably expressed the VEGF receptor-2 were transiently transfected to express the KAPh SH2 domain or full-length KAPh were treated with 1 nM VEGF or left untreated for 5 min. The cells were lysed in 1% triton lysis buffer with protease inhibitors. Insoluble debris was removed from the lysates by centrifugation at 14,000×g for 10 min at 4° C. The protein extracts were subjected to immunoprecipitation with anti-VEGFR2 antibody (R2.2) and protein A/G agarose (Santa Cruz Biotechnology, Inc., Santa Cruz, Calif.). The immunoprecipitated proteins were resolved by SDS-PAGE, transferred to PVDF, and probed with antibodies to either the 6×HisG epitope (Invitrogen, Carlsbad, Calif.) or to KAPh (62507 a.p.). The blots were subsequently stripped and reprobed with anti-VEGFR2.antibody (R2.2) to determine equal loading.

Northern Blot Analysis of KAPh Transcript

A 12-lane human multiple tissue Northern blot was obtained from Clontech (Palo Alto, Calif.). Likewise, a 12-lane mouse multiple tissue Northern blot and a mouse embryo Northern blot were obtained from OriGene Technologies, Inc. (Rockville, Md.). A cDNA probe for KAPh was labeled with [α-³²P]dCTP using a RediPrime II random prime labeling system (Amersham Pharmacia, Piscataway, N.J.). Each blot was hybridized with the labeled probe in ExpressHyb hybridization solution (Clontech, Palo Alto, Calif.) and were processed according to the manufacturer's instructions. Each blot was subsequently re-probed with ³²P-labeled α-actin for normalization.

Immunohistochemistry

The mouse monoclonal antibody, MoAb33, which was raised against the extracellular domain of the human Tie2 protein, was used as a marker for rat endothelium(Wong A L, et al., Circ Res 1997; 81(4):567-574). The affinity purified rabbit polyclonal antibody (62507 a.p.) was used to detect KAPh expression. Snap-frozen sections of rat heart and rat kidney tissue were treated in 0.3% hydrogen peroxide/MeOH for 30 min, blocked in 10% normal horse or donkey serum for 1 h, and blocked with biotin/avidin for 15 min each. The sections were then incubated with either MoAb33 or 62507 a.p. primary antibody for 1 h at room temperature or overnight at 4° C., biotinylated horse anti-mouse (MoAb33) or biotinylated donkey anti-rabbit (62507 a.p.) for 1 h, and Vector Elite ABC biotin-avidin-peroxidase complex (Vector Laboratories, Burlingame, Calif.) for 30 min. The sections were then developed with diaminobenzidine, counterstained with hematoxylin, and coverslipped.

Determination of Phosphatase Activity

Purified, recombinant KAPh phosphatase domain (amino acids 83-464 SEQ ID NO 4) (100 μg/mL) and full-length PTEN (amino acids 1-403) (100 μg/mL) were each mixed with a fluorogenic phosphatase substrate 6,8-difluoro-4-methylumbelliferyl phosphate (DiFMUP) (Molecular Probes, Eugene, Oreg.) at a final concentration of a 100 μM in assay buffer (10 mM sodium acetate, pH 6.0, 150 mM NaCl, 5 mM DTT). Following incubation at ambient temperature for 15 or 60 min., the fluorescence intensity of the 6,8-difluoro-4-methylumbelliferone (DiFMU) product was measured in triplicate and used as an assessment of phosphatase activity. A Victor 5 microplate reader from Wallac (Turku, Finland) was used to read the fluorescence intensity from 384-well microplates (Corning Inc., Corning, N.Y.).

Results

In an ongoing effort to understand mechanisms by which the biological activity of VEGF is mediated through the VEGF receptor-2 (KDR/Flk-1), we used the entire cytoplasmic domain of KDR as bait to screen human umbilical vein endothelial cell (HUVEC) and human fetal brain cDNA libraries in the yeast two-hybrid system. The intracellular domain of human VEGFR2 (790-1357 SEQ ID NO 8) was constructed in the bait plasmid (pDBLeu) and the cDNA libraries were constructed in the prey plasmid (pPC86). Following transformation of yeast, positive clones were selected by growth on histidine-deficient media and for the expression of β-galactosidase activity (X-gal selection). Subsequently, plasmid DNAs isolated from yeast harboring putative interacting clones were sequenced to determine their identity, then plasmids of a select number of clones were re-transformed into yeast in combination with various specificity controls.

Yeast transformed with plasmids encoding interacting clones along with vector controls were picked and patched onto histidine-containing media to confirm the expression of each plasmid pair (FIG. 1A). The growth properties of one clone, designated KAPh for KDR-Associated Phosphatase, that was identified as a KDR-interacting protein from both cDNA libraries are shown in FIG. 1. Yeast containing wild-type KDR and the interacting domain of KAPh showed robust protein-protein interaction, evident by growth on histidine-deficient (FIG. 1B, row 3) and uracil-deficient (FIG. 1C, row 3) media. In contrast, there was essentially no significant interaction of KAPh with the pDBLeu parent vector (FIG. 1B, row 2; FIG. 1C, row 2) or with a mutant form of KDR in which the ATP binding site had been mutated (K870R) (FIG. 1B, row 4; FIG. 1C, row 4). Likewise, no discernable interaction was obtained with any other combinations of yeast bait and prey control plasmids (FIG. 1B, rows 1, 5-6; FIG. 1C, rows 1,5-6). To further confirm these yeast two-hybrid interactions we examined the ability of yeast expressing the above described plasmid combinations to activate a third integrated reporter gene, LacZ. As shown in FIG. 1D, by spectrophotometrically measuring the β-galactosidase activity in liquid cultures of yeast transformed with each combination of plasmids, it is clear that the interaction of KAPh is specific for the wild-type VEGF receptor-2.

In Vitro Association of KAPh with KDR. To confirm the yeast two-hybrid data, interactions between the KAPh interaction domain and KDR were examined in vitro by glutathione S-transferase (GST) pull-down analyses. Purified, baculovirus expressed GST, wild-type GST VEGFR2, and catalytically inactive GST VEGFR2 K870R were combined with HEK 293 cell lysate that transiently expressed hexahistidine-tagged KAPh SH2 domain. As illustrated in FIG. 2A, the KAPh SH2 domain-containing protein fragment interacted in an autophosphorylation-dependent manner with the VEGF receptor-2 (FIG. 2A, lanes 5 and 7), while no interaction was observed with GST alone (FIG. 2A, lane 3). The immunoblot was sequentially stripped of antibody and re-probed with anti-GST and anti-phosphotyrosine antibodies to determine relative amounts of each GST fusion protein (note: GST protein, which ran at ˜26 kDa, is not shown) and that only the wild-type but not the catalytically inactive VEGFR2 was tyrosine phosphorylated.

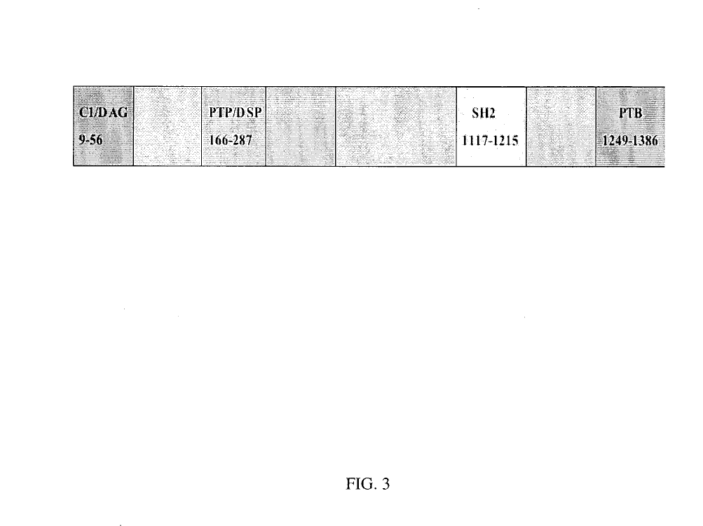

Following verification of the protein-protein association between KDR and the interacting fragment of KAPh (KAPh SH2 domain) that was obtained from the yeast two-hybrid screen, we cloned the full-length KAPh protein. The isolation of a cDNA clone encoding full-length KAPh and the identification of functional domains contained within the full-length amino acid sequence are described in FIG. 3.

The cDNA encoding full-length KAPh was subcloned into the pcDNA4/HisMax mammalian expression vector to transiently express full-length KAPh in HEK 293 cells. The associations of KAPh with VEGFR2 or other endothelial cell receptor tyrosine kinases were examined in vitro by GST pull-down analyses. Purified, baculovirus expressed GST, wild-type and catalytically inactive mutants of human VEGFR-1, VEGFR2, Tie 1, or Tie 2 were combined with HEK 293 cell lysate that transiently expressed hexahistidine-tagged full-length KAPh. As illustrated in FIG. 2B, full-length KAPh interacted in an autophosphorylation-dependent manner with the VEGF receptor-2 (FIG. 2B, compare lanes 5 and 6), while no interaction was observed with GST alone (FIG. 2B, lane 2). Full-length KAPh also showed weak, but reproducible, autophosphorylation-dependent interaction with VEGF receptor-1 (compare lanes 3 and 4). However, the relative strength of interaction of KAPh with VEGFR-1 was significantly less than that of KAPh with VEGFR2 (FIG. 2B, lanes 3 vs. 5), particularly when corrected for protein levels by anti-GST blot and levels of autophosphorylation by anti-pTyr blot. Interestingly, KAPh showed no interaction with other human endothelial cell receptor tyrosine kinases examined, specifically Tie 1 and Tie 2 (FIG. 2B, lanes 10 and 11, lanes 12 and 13), even though both showed some degree of tyrosine phosphorylation.

Since our yeast two-hybrid and in vitro interaction data suggested that KAPh was interacting with the VEGF receptor-2 in an autophosphorylation-dependent manner, we generated tyrosine to phenylalanine (Y→F) mutants of multiple residues within the cytoplasmic domain of KDR that have previously been identified as autophosphorylation sites or have been shown to be docking sites for other interacting proteins. Each Y→F mutant was generated as a GST fusion protein in the baculovirus expression system and examined for the ability to associate with full-length KAPh in pull-down experiments. In vitro interaction data for some of the mutants tested are illustrated in FIG. 2C. As was shown in FIG. 2B, we observed autophosphorylation-specific association of full-length KAPh and the VEGF receptor-2 (FIG. 2C, compare lanes 2 and 3). We detected no significant change in association of KAPh with the Y951F mutant (FIG. 2C, lane 4). In contrast, mutation of tyrosine 1175 of KDR (Y1175F) nearly abolished its interaction with KAPh. Importantly, these differences were not a result of alterations in protein levels (anti-GST blot) or levels of autophosphorylation (anti-pTyr blot). These data indicate that tyrosine 1175 of KDR is essential for association with KAPh.

Co-immunoprecipitation of full-length KAPh with full-length VEGF receptor-2. To further verify the association of KAPh and VEGF receptor-2 we examined immunoprecipitates from lysate of VEGFR2 expressing cells for the presence of full-length KAPh. When both proteins were co-expressed and immunoprecipitated using a polyclonal antibody specific for VEGFR2, a band corresponding to the size of full-length KAPh is detected in cells stimulated with VEGF (FIG. 2D, compare lanes 5 and 6). This band is not apparent in immunoprecipitates of VEGFR2 stable cells that were transiently transfected with pcDNA4/HisMax A empty vector, irrespective of VEGF stimulation (FIG. 2D, lanes 2 and 3). Importantly, the band corresponding to full-length KAPh was also not detected in mock immunoprecipitates (where no VEGFR2 primary antibody was added) (FIG. 2D, lane 4), eliminating the possibility that full-length KAPh was non-specifically binding to protein A/G agarose beads. These data demonstrate that the KDR-KAPh complex can indeed form, but only in cells that have been stimulated with VEGF. Taken together these findings suggest that KAPh association with KDR requires autophosphorylation of the receptor and the exposure of a docking site (phosphorylated Tyr 1175 of SEQ ID NO 2) for binding the SH2 domain-containing protein KAPh.

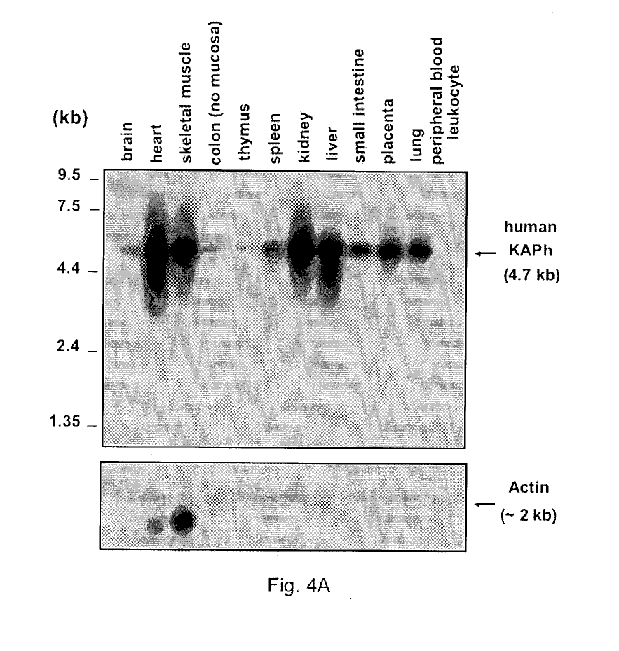

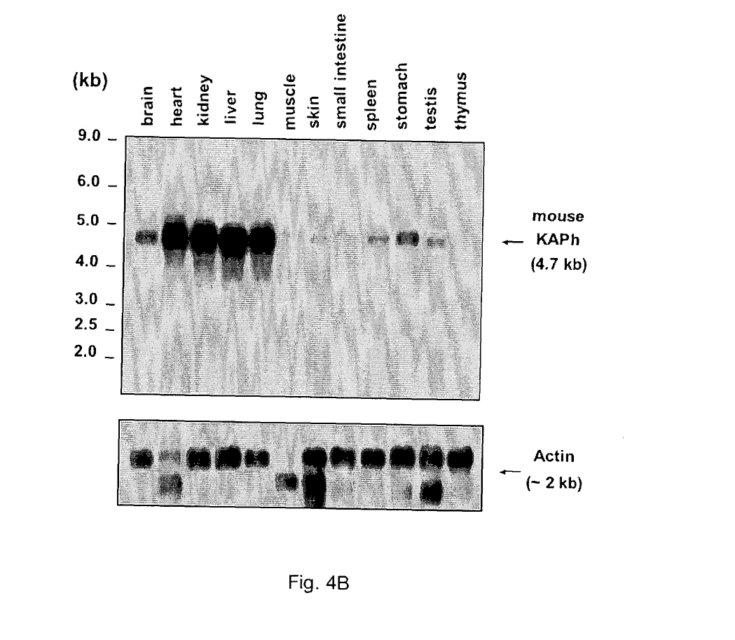

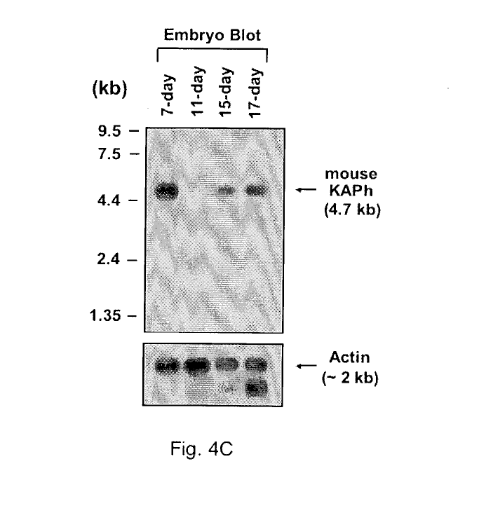

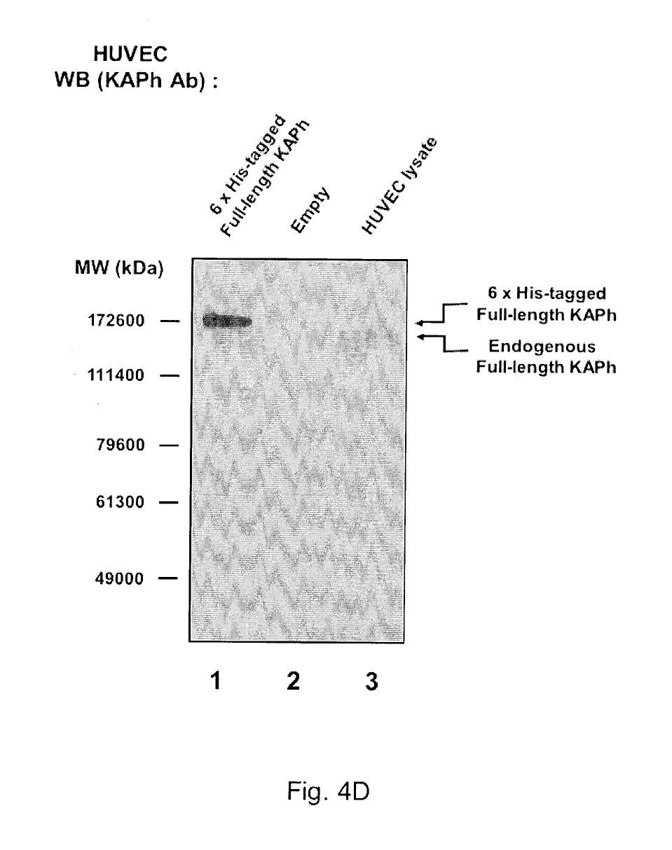

Characteristics of the full-length KAPh cDNA, mRNA transcript, and expressed protein. The plasmid encoding the interacting protein fragment of KAPh isolated from the yeast two-hybrid screen was sequenced and found to be a novel cDNA that contained a Src homology 2 (SH2) domain. In order to obtain a cDNA clone encoding the full-length KAPh protein a Rapid-Screen arrayed cDNA library (OriGene Technologies, Rockville, Md.) was screened by PCR. From the screen of a human heart cDNA library master plate and subplates using primers specific for KAPh, we obtained a single plasmid that encodes the entire KAPh cDNA. We isolated a 4688-bp cDNA consisting of a noncoding leader sequence of 18 bp, a 4158-bp open reading frame, and 512 bp of 3′-noncoding sequence (FIG. 3). The size of this cDNA is consistent with Northern blot analysis which revealed a single ˜4.7 kb mRNA transcript for human KAPh (FIG. 4A).

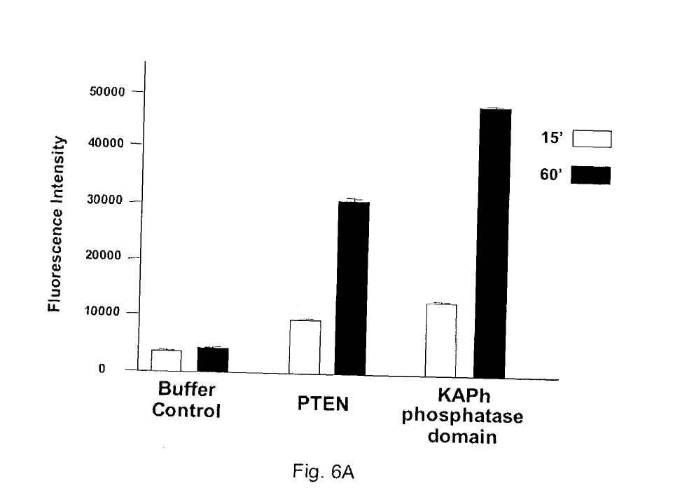

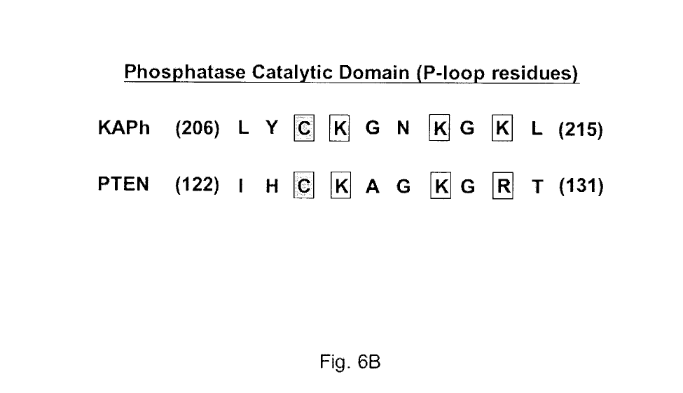

A bioinformatics approach was then employed to determine regions of the KAPh protein that are important for its interaction with the VEGF receptor-2 and to identify other protein motifs that may provide insight into the functional importance of KAPh and the significance of its interaction with VEGFR2. The deduced amino acid sequence of full-length KAPh, illustrated in FIG. 3, contains several important functional domains. Those residues comprising the interacting protein fragment isolated from the yeast two-hybrid screen are underlined. The SH2 domain contained within the interacting fragment is underlined and highlighted in bold. Another potentially interesting region identified within the amino acid sequence of full-length KAPh is a domain important for binding diacylglycerol/phorbol ester (C1 domain) (amino acids 9 to 56 of SEQ ID NO 2). KAPh also contains a domain with very high homology to the catalytic region of protein tyrosine phosphatases/dual-specificity phosphatases (PTP/DSP) (SEQ ID NO 4). The phosphatase activity of KAPh has been experimentally determined and the results are presented in FIG. 6.

The tissue distribution of KAPh was determined by Northern blot analysis using a human multiple tissue blot (Clontech, Palo Alto, Calif.). The ˜4.7 kb KAPh mRNA transcript was present in poly(A)⁺ RNA from nearly all human tissues examined with strongest expression in highly vascularized tissues including heart, kidney, liver, lung, skeletal muscle, brain (FIG. 4A). The blot was stripped and rehybridized with a β-actin probe to evaluate the relative amounts of RNA on the blot. Subsequently, in order to confirm this exciting pattern of expression we obtained an IMAGE clone (Research Genetics, Huntsville, Ala.) corresponding to the mouse homologue of KAPh for use as a cDNA probe of a mouse multiple tissue blot (OriGene Technologies, Rockville, Md.). Northern blot analysis using a mouse KAPh cDNA probe (SEQ ID NO 9) yielded a single ˜4.7 kb transcript with a very similar pattern of expression, highest in heart, kidney, liver, and lung (FIG. 4B). This blot was also stripped and rehybridized with a β-actin probe to evaluate the relative amounts of RNA on the blot.

To determine whether KAPh was expressed in endothelial cells, a polyclonal antibody that specifically recognizes KAPh was used to examine the expression of the full-length KAPh protein in HUVEC lysate. A specific band with an apparent molecular weight of ˜160 kDa (FIG. 4D, lane 3) corresponding to endogenous KAPh present in endothelial cells was detected. The KAPh antibody also recognized overexpressed, epitope-tagged full-length KAPh (FIG. 4D, lane 1). As expected, due to the presence of the epitope, recombinant HisMax-tagged KAPh appeared to migrate as a slightly larger protein.

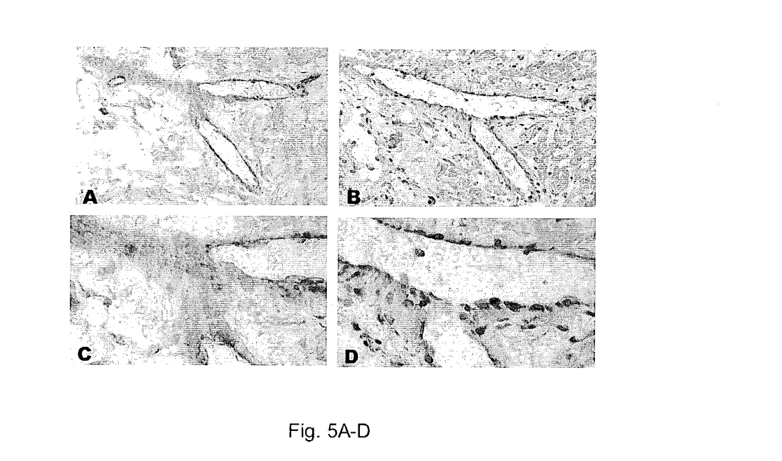

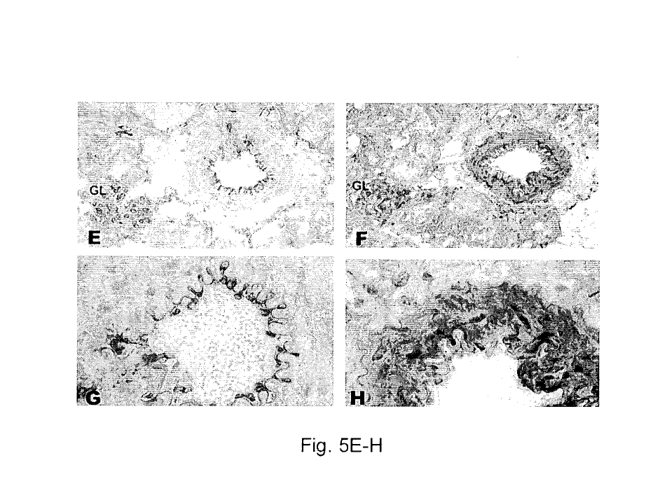

Since KAPh was isolated from a human umbilical vein endothelial cell cDNA library and the full-length protein was detected in endothelial cell lysate by immunoblot using a KAPh-specific antibody, we wanted to further examine its expression by immunohistochemistry (IHC). To determine whether KAPh expression was restricted to the vascular endothelium, multiple adult rat tissue sections were screened by IHC. In all tissues examined (heart, kidney, skeletal muscle, liver, lung, brain), KAPh was strongly expressed throughout the vasculature in both the endothelium of arteries, veins, and capillaries as well as surrounding smooth muscle of larger vessels. In rat heart, immunostaining of KAPh protein in a longitudinal cross-section of a bifurcating blood vessel clearly showed strong expression of KAPh in the vascular endothelium (FIGS. 5, B and D). Importantly, comparison with IHC of rat heart using rabbit pre-immune serum demonstrated that the labeling that we observed was specific (data not shown). Furthermore, we feel quite certain that KAPh is expressed in the vascular endothelium since similar labeling was observed with an anti-Tie 2 antibody, a positive marker for rat endothelium (FIGS. 5, A and C). Likewise, immunostaining of KAPh protein in rat kidney showed strong expression in the vascular endothelium and surrounding smooth muscle of larger blood vessels as well as in fenestrated glomerular capillaries (FIGS. 5, F and H). Again, comparison with rabbit pre-immune serum (data not shown) or Tie 2 (FIGS. 5, E and G) demonstrated that the labeling we observed was specific. Taken together these data show that, at both the mRNA and protein levels, KAPh is predominantly expressed in the vascular endothelium and surrounding smooth muscle of blood vessels in highly vascularized tissues and is present cultured vascular endothelial cells.

KAPh has phosphatase activity against a small fluorogenic substrate. Having demonstrated an association between KAPh and the VEGF receptor-2 both in yeast and in vitro and the expression of KAPh in the vasculature, the functional importance of KAPh was then examined. Phosphatase assays were conducted using purified, baculovirus expressed KAPh protein and a fluorogenic phosphatase substrate (DiFMUP). As illustrated in FIG. 6, the KAPh phosphatase domain exhibited a time-dependent increase in phosphatase activity. The fluorescent product (DiFMU) generated following a 60 min. incubation of KAPh with DiFMUP was nearly 10-fold greater than buffer control (FIG. 6A). Upon closer inspection of the phosphatase catalytic domain (P-loop residues) of KAPh we found that it shared a number of conserved residues with PTEN, a tumor suppressor with phosphatase activity (FIG. 6B). Namely, both proteins contained a critical invariant cysteine (KAPh, Cys 208 SEQ ID NO 2; PTEN, Cys 124) and both had a preponderance of basic residues in the +1, +4, and +6 positions. By comparison, the incubation of PTEN with DiFMUP showed a 6-fold increase in generation of fluorescent DiFMU product (FIG. 6A). These findings suggest that KAPh possesses phosphatase activity that is comparable to PTEN and based on the conservation of amino acid residues within the catalytic phosphatase domain of each protein it is possible that they may have similar functions and act by dephosphorylating a common pool of substrates.

Discussion of Results

As discussed above, biological activity of VEGF is mediated, by signal transduction pathways downstream of the VEGF receptor-2 (KDR/Flk-1). Although several studies have now reported the identification and characterization of some of these important signaling intermediates, we believe that other known and possibly even some novel signaling proteins that associate with the KDR have yet to be identified. The present study demonstrates that the KDR associates with a novel protein, KAPh, in an autophosphorylation-dependent manner bringing a newly identified interacting protein with phosphatase activity to the receptor. Further characterization of KAPh revealed that it is expressed, both at the mRNA and protein levels, in the vascular endothelium and smooth muscle of blood vessels of highly vascularized tissues. Taken together these findings suggest that KAPh may be an important signaling intermediate downstream of the VEGF receptor-2 and that it may play a role in modulating the activity of the receptor.

Activation of its intrinsic receptor tyrosine kinase activity by binding VEGF plays an important role in VEGF receptor-2 (KDR/Flk-1) autophosphorylation and its subsequent association with cytoplasmic signaling proteins. Based on our protein-protein interaction data, the association of KDR and KAPh is dependent on activation and tyrosine phosphorylation of the receptor (FIGS. 1 and 2). Autophosphorylation-dependent interactions between KDR and other cytoplasmic signaling molecules including phospholipase Cy (PLCy), a Shc-related adaptor protein (Sck), and a low molecular weight protein tyrosine phosphatase (HCPTPA), have also been reported (Cunningham S A, et al., Biochem Biophys Res Commun 1997; 240(3):635-639; Igarashi K, et al., Biochem Biophys Res Commun 1998; 251(1):77-82; Warner A J, et al., Biochem J 2000; 347(Pt 2):501-509; Huang L, et al., J Biol Chem 1999; 274(53):38183-38188). Mutational analysis of potential tyrosine autophosphorylation sites on KDR identified tyrosine 1175 as a critical residue for binding the SH2 domain of both PLCγ and Sck. Interestingly, we found tyrosine 1175 to be essential for the interaction of KDR with KAPh (FIG. 2C). In contrast, the association of KDR and VRAP, a VEGF receptor-associated protein, appeared to be constitutive and the level of association was not enhanced by stimulation with VEGF (Wu L W, et al., J Biol Chem 2000; 275(9):6059-6062). Moreover, the interaction of KDR with VRAP was disrupted by mutation of tyrosine 951, but was unaffected by Y1175F mutation. It now appears that there are at least two confirmed autophosphorylation sites on KDR for binding SH2 domain-containing signaling proteins. The functional significance of the interaction of KDR with either Sck or PLCγ has not yet been fully determined so it is difficult to speculate on the role KAPh may have in competing for binding to the Tyr 1175 site, thereby reducing the activities of these other KDR-associated proteins. Obviously, further studies of all of these potentially important physiological effectors will be required to fully understand their activities and how their interplays regulate VEGF signal transduction.

The nucleotide sequence of human KAPh contains a partial cDNA clone (KIAA1075) that was isolated as an unidentified human gene from a fetal brain cDNA library (Kikuno R, et al., DNA Res 1999; 6(3):197-205). The expression of this gene in brain was confirmed by our isolation of KAPh from both a human fetal brain and a vascular endothelial cell cDNA library, as well as our Northern blot analysis showing the expression of the KAPh mRNA transcript in human brain poly(A)+RNA (FIG. 4A). The deduced 1386-amino acid sequence of KAPh is very similar (43.1% identical, residues 1-1386) to human tensin, a focal adhesion molecule. Tensin was first identified as a SH2 domain-containing cytoskeletal protein that binds and caps actin filaments at focal contacts (Davis S, et al., Science 1991; 252(5006):712-715; Lo S H, et al., J Biol Chem 1994; 269(35):22310-22319; Lo S H, et al., Bioessays 1994; 16(11):817-823; Chen H, et al., Biochem J 2000; 351 Pt 2:403-411; Lo S H, et al., J Cell Biol 1994; 125(5):1067-1075). Multiple sequence alignments of KAPh and human tensin revealed that these proteins have even higher similarity at their extreme carboxyl-termini (65.7% identical, residues 1106-1386 of SEQ ID NO 2), most likely due to the presence of SH2 domain residues. Interestingly, these proteins share another region of high homology near their amino-termini (53.1% identical, residues 97-545). Even though this region overlaps two of the three actin-binding domains in tensin, whether KAPh has the ability to directly bind actin filaments has yet to be determined. Also contained within this amino-terminal region is a domain with a high degree of homology to protein tyrosine phosphatase/dual-specificity phosphatases (PTP/DSP) (Haynie DT, Ponting CP, Protein Sci 1996; 5(12):2643-2646). However, after closer inspection of amino acid residues forming the putative catalytic phosphatase domain, we believe that it is very unlikely that tensin possesses phosphatase activity. This prediction is strongly supported by the observation that an invariant nucleophilic cysteine residue is mutated to asparagine (C113N) in tensin. Importantly, KAPh retains a cysteine residue (Cys 208) at this position.

The amino-terminal region of KAPh also shares homology (34.7% identity, residues 108-269 of SEQ ID NO 2) with PTEN, a recently identified tumor suppressor protein. PTEN [phosphatase and tensin homologue (PTEN)/mutated in multiple advanced cancers (MMACI)/TGFβ-regulated and epithelial cell-enriched phosphatase (TEP1)] was originally identified as a candidate tumor suppressor with sequence similarity to protein tyrosine phosphatases (PTPs) and tensin (Li J et al., Science 1997; 275(5308): 1943-1947; Steck P A, et al., Nat Genet 1997; 15(4):356-362; Li D M et al., Cancer Res 1997; 57(11):2124-2129; Li D M et al., Proc Natl Acad Sci U S A 1998; 95(26):15406-15411). More recently, the biological activities of PTEN have begun to be determined and it has now been shown that PTEN possesses phosphatase activity against a specific, well-defined pool of substrates. One of the most important physiological substrates of PTEN identified thus far is the phosphoinositide second messenger phosphatidylinositol 3,4,5-trisphosphate (PIP₃) (Maehama T, et al., J Biol Chem 1998; 273(22):13375-13378). PTEN acts by dephosphorylating the D3 position of PIP₃ and appears to functionally oppose the activities of phosphatidylinositol 3-kinase (PI3K). Several studies and many review articles have highlighted the important role that PTEN is thought to play in regulating signal transduction pathways involved in cell growth and migration (See e.g., Myers M P, et al., Proc Natl Acad Sci U S A 1997; 94(17):9052-9057; Tamura M, et al., Science 1998; 280(5369):1614-1617; Myers M P, et al., Proc Natl Acad Sci U S A 1998; 95(23):13513-13518; Cantley L C, et al., Proc Natl Acad Sci U S A 1999; 96(8):4240-4245; Maehama T, et al., Trends Cell Biol 1999; 9(4):125-128). The catalytic phosphatase domains of PTEN and KAPh appear to quite similar both at the primary amino acid sequence level (FIG. 6B) and in a three-dimensional homology model that was built based on the crystal structure of PTEN (Lee J O, et al., Cell 1999; 99(3):323-334), See FIG. 7. Overall, our homology model suggests that KAPh should act as a phosphatase. However, due to a T215L substitution the rate of hydrolysis of the phospho-enzyme intermediate may be significantly reduced.

Based on the high degree of homology between the catalytic phosphatase domains of PTEN and KAPh, we assayed KAPh for phosphatase activity. Using a fluorogenic phosphatase substrate (DiFMUP), we observed detectable phosphatase activity by KAPh that was nearly 10-fold greater than the buffer control by 1 h (FIG. 6A). Furthermore, the phosphatase activity exhibited by KAPh against DiFMUP was 1- to 2-fold greater than that of PTEN. Despite activity against DiFMUP, we were unable to detect significant, reproducible phosphatase activity for either KAPh or PTEN using any peptide substrates examined (data not shown). However, it is possible that the phosphatase activity exhibited by KAPh was below the limit of detection in the considerably less sensitive BIOMOL green phosphatase assay employed for in the peptide phosphatase assay. We were also unable to detect a change in phosphorylation of ³²P-labeled VEGF receptor-2 when incubated overnight in the presence of KAPh (data not shown). Therefore, we believe that KAPh most likely does not possess protein tyrosine phosphatase (PTP) activity. Experiments designed to determine whether KAPh is able to dephosphorylate lipid phosphate substrates (e.g., inositol phosphates and phosphatidylinositol phosphates) are in progress.

It is well established that angiogenic growth factor receptors, in particular VEGF receptors, are highly expressed in the vasculature. Using Northern blot, Western blot, and immunohistochemistry (IHC) analyses we examined the expression of the novel KDR associated protein KAPh. Northern blot analyses of both human and mouse multiple tissue blots revealed that KAPh expression appeared to be strongest in highly vascularized tissues including heart, kidney, liver, lung, skeletal muscle, and brain (FIGS. 4A and B). An identical pattern of expression for human KAPh was observed when the human blot was probed again using a second non-overlapping KAPh cDNA probe (data not shown).

Western blot analysis of human umbilical vascular endothelial cells (HUVEC) using a polyclonal anti-KAPh antibody clearly demonstrated the expression of the full-length KAPh protein in the vascular endothelium (FIG. 4D). The expression of KAPh in the human endothelium was substantiated by St. Croix et al. in which the identification of an uncharacterized protein KIAA1075/PEM10 (tenth most abundant novel pan endothelial marker) present in both normal (N-ECs) and tumor endothelial cells (T-ECs) as well as cultured HUVEC and human microvascular endothelial cells (HMVEC) was reported (St Croix B, et al., Science 2000; 289(5482):1197-1202). It is important to note that the entire partial cDNA clone KIAA1075 is contained within the cDNA clone encoding the full-length KAPh protein. To extend these observations and to determine if the expression of KAPh was restricted to the vascular endothelium, we examined the expression of KAPh in multiple adult rat tissues by immunohistochemistry (IHC) using our anti-KAPh antibody. In all tissues examined (heart, kidney, skeletal muscle, liver, lung, brain), KAPh was strongly expressed throughout the vasculature in both the endothelium of arteries, veins, and capillaries as well as surrounding smooth muscle of larger vessels (FIG. 5, panels B, D, F, and H). The specificity of this labeling was confirmed using rabbit pre-immune serum (data not shown). Taken together these data show that, at both the mRNA and protein levels, the expression of KAPh is restricted to the vascular endothelium and surrounding smooth muscle of blood vessels in highly vascularized tissues and is present cultured vascular endothelial cells.

References incorporated herein by reference.

What is claimed is:

1. An isolated polypeptide of SEQ ID NO 2 or variant thereof.

2. An isolated nucleotide of SEQ ID NO 1 or variant thereof.

3. A method of using KAPh, or variant thereof, in the treatment of an angiogenesis mediated disorder in a subject in need thereof.

4. The method of claim 3, wherein the angiogenesis mediated disorder is selected from the group consisting of cancer, diseases associated with retinal/choroidal neovascularization, diseases associated with rubeosis, diseases caused by abnormal proliferation of fibrovascular or fibrous tissue, diseases associated with chronic inflammation, inflammatory bowel diseases, rheumatoid arthritis, and diseases associated with ischemic tissue or damage

5. A method of screening an agent useful for treating an angiogenesis mediated disorder comprising the steps of:
a. exposing KAPh, or variant thereof, to the agent; and
b. measuring activity of KAPh;
wherein a modulation in KAPh activity indicates the agent is useful for treating the angiogenesis mediated disorder.

6. The method of claim 5, wherein KAPh is selected from SEQ ID NO 2 or 4.

7. The method of claim 6, wherein measuring activity of KAPh comprises measuring phosphatase activity.

8. The method of claim 7, wherein measuring activity of KAPh comprises measuring fluorescence of 6,8-difluoro-4-methylumbelliferyl phosphate (DiFMUP).

9. The method of claim 8, wherein the agent is selected from the group consisting of peptide, peptidomimetic, polypeptide, protein, chemical compound, nucleotide, antibody, small molecule, vitamin derivative and carbohydrate.

10. A method of screening an agent useful for modulating angiogenesis comprising the steps of:
a. exposing KAPh, or variant thereof, to the agent;
b. exposing KAPh to VEGFR2; and
c. measuring association of KAPh and VEGFR2;
wherein a modulation is the association of KAPh and VEGFR2 indicates the agent is useful for treating the angiogenesis mediated disorder.

11. The method of claim 10, wherein VEGFR2 is selected from SEQ ID NOs 6 or 8.

12. The method of claim 11, wherein the method of measuring association of KAPh and VEGFR2 is selected for the group coimmunoprecipitation, pull-down assay, and yeast-two hybrid system

13. A method of screening an agent useful for treating an angiogenesis mediated disorder comprising the steps of:
a. exposing a cell to the agent; and
b. measuring activity of KAPh in the cell;
wherein a modulation in the expression or the activity of KAPh indicates the agent is useful for treating the angiogenesis mediated disorder.

14. The method of claim 13, wherein the cell is selected from the group consisting of liver, lung, skeletal muscle, and brain.

15. A method of screening an agent useful for treating an angiogenesis mediated disorder comprising the steps of:
a. exposing a cell to the agent; and
d. measuring association of KAPh and VEGFR2;
wherein a modulation is the association of KAPh and VEGFR2 indicates the agent is useful for treating the angiogenesis mediated disorder.

16. A method of screening an agent useful for treating an angiogenesis mediated disorder comprising the steps of:
c. exposing a cell to the agent; and
d. measuring expression of KAPh in the cell;
wherein a modulation in the expression or the activity of KAPh indicates the agent is useful for treating the angiogenesis mediated disorder.

17. An antibody binding KAPh or variant thereof.
